(19) United States
(12) Patent Application Publication
(10) Pub. No.: US 2008/0102311 A1
(43) Pub. Date: May 1, 2008

(54) MATERIAL FOR ORGANIC ELECTROLUMINESCENT ELEMENT AND ORGANIC ELECTROLUMINESCENT ELEMENT EMPLOYING THE SAME

(75) Inventor: Masakazu Funahashi, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Idemitsu Kosan Co., Ltd., Chiyoda-ku (JP)

(21) Appl. No.: 11/833,550

(22) Filed: Aug. 3, 2007

(30) Foreign Application Priority Data
Aug. 4, 2006 (JP) 2006-213328

(51) Int. Cl. H01J 1/62; C07D 209/82; C07D 241/42; C07F 7/10; C07C 211/54

(52) U.S. Cl. 428/690; 564/434; 556/413; 564/429; 564/427; 544/353; 548/442

(57) ABSTRACT

A material for electroluminescence devices comprising an aromatic amine derivative having a specific structure having bulky substituents at end portions and an organic electroluminescence device comprising an organic thin film layer which comprises a single layer or a plurality of layers comprising at least a light emitting layer and is disposed between an anode and a cathode, wherein at least one layer in the organic thin film layer comprises the material for organic electroluminescence devices singly or as a component of a mixture. The electroluminescence device having a long life and exhibiting a great luminance of emitted light and a great efficiency of light emission can be obtained by using the material.

### TECHNICAL FIELD

The present invention relates to a material for electroluminescence (hereinafter, “electroluminescence” will be referred to as “EL”) devices and an organic EL device using the material and, more particularly, to an organic EL device having a long life and exhibiting a great luminance of emitted light and a great efficiency of light emission and a material for organic EL devices enabling to obtain the device.

### BACKGROUND ART

Organic EL devices using organic substances are expected to be useful as the inexpensive full color display device of the solid light emission type having a great area, and various developments have been made. In general, an EL device is constituted with a light emitting layer and a pair of electrodes disposed at both sides of the light emitting layer. For the light emission, electrons are injected at the side of the cathode, and holes are injected at the side of the anode when an electric field is applied. The electrons are combined with the holes in the light emitting layer to form excited states, and the energy formed when the excited states returns to the ground state is discharged as light.

Conventional organic EL devices require greater driving voltages and exhibit smaller luminances of emitted light and smaller efficiencies of light emission than those of inorganic light emitting diodes. Moreover, marked deterioration in the properties takes place, and the devices have not been used in practical applications. The properties of the organic EL devices are being improved gradually, but a greater efficiency of light emission and a longer life are required.

For example, a technology in which a single anthracene compound is used as the organic light emitting material is disclosed (Patent Reference 1). However, since the luminance is as small as 1,650 cd/m² at a current density of 165 mA/cm², and the efficiency is as small as 1 cd/A in accordance with this technology, this technology cannot be used in the practical applications. A technology in which a single bisanthracene compound is used as the organic light emitting material is disclosed (Patent Reference 2). However, the efficiency is as small as about 1 to 3 cd/A in this technology, and improvement is required for the practical application. An organic EL device having a long life in which a distyryl compound is used as the organic light emitting material and styrylamine is added to the distyryl compound, is proposed (Patent Reference 3). However, the obtained device does not have a sufficient life, and further improvement is required.

In Patent Reference 4, a material for organic EL devices in which end portions are each substituted with substituents having benzene ring is described. The material for organic EL devices is decomposed during preparation of the device since vapor deposition of the material requires a high temperature.

[Patent Reference 1] Japanese Patent Application Laid-Open No. Heisei 11 (1999)-3782

[Patent Reference 2] Japanese Patent Application Laid-Open No. Heisei 8 (1996)-12600

[Patent Reference 3] International Patent Application Laid-Open No. WO 94/006157

[Patent Reference 4] Japanese Patent Application Laid-Open No. Heisei 10 (1998)-251633

### DISCLOSURE OF THE INVENTION

The present invention has been made to overcome the above problems and has an object of providing an EL device having a long life and exhibiting a great luminance of emitted light and a great efficiency of light emission and a material for organic EL devices enabling to obtain the device.

As the result of intensive studies by the present inventors to achieve the above object, it was found that the object could be achieved by utilizing as the material for organic EL devices an aromatic amine compound represented by the following general formula (1) or (2) which has bulky substituents at end portions. The present invention has been completed based on the knowledge.

The present invention provides a material for organic electroluminescence devices which comprises an aromatic amine derivative represented by following general formula (1) or general formula (2): 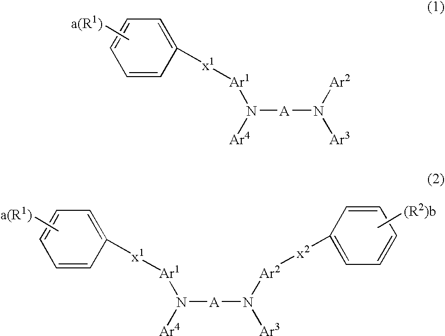
wherein

A represents a substituted or unsubstituted aromatic hydrocarbon cyclic group having 6 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;

Ar¹ to Ar⁴ each independently represent a substituted or unsubstituted aromatic hydrocarbon cyclic group having 6 to 40 nuclear carbon atoms or a substituted or unsubstituted aromatic heterocyclic group having 5 to 40 nuclear carbon atoms, wherein Ar¹ represents a divalent group, Ar² represents a monovalent or a divalent group and Ar³ and Ar⁴ each represent a monovalent group;

X¹ to X⁴ each independently represent —O—, —S—, >C═O, >SO₂, —(CₓH₂ₓ)—O—(C_{y}H_{2y})—, x and y each representing an integer of 0 to 20 excluding a case where x+y=0, a substituted or unsubstituted alkylidene group having 2 to 20 carbon atoms, a substituted or unsubstituted alkylene group having 2 to 20 carbon atoms or a substituted or unsubstituted divalent aliphatic cyclic group having 3 to 10 nuclear carbon atoms;

R¹ and R² each independently represent hydrogen atom, a substituted or unsubstituted alkyl group having 1 to 50 carbon atoms, a substituted or unsubstituted aryl group having 5 to 50 nuclear carbon atoms, a substituted or unsubstituted aralkyl group having 6 to 50 nuclear carbon atoms, a substituted or unsubstituted cycloalkyl group having 3 to 50 nuclear carbon atoms, a substituted or unsubstituted alkoxyl group having 1 to 50 carbon atoms, a substituted or unsubstituted aryloxyl group having 5 to 50 nuclear carbon atoms, a substituted or unsubstituted arylamino group having 5 to 50 nuclear carbon atoms, a substituted or unsubstituted alkylamino group having 1 to 20 carbon atoms or a substituted or unsubstituted heterocyclic group having 5 to 50 nuclear carbon atoms; and

a and b each represent an integer of 1 to 5 and, when any of a and b represents an integer of 2 or greater, groups in ( ) may be same with or different from each other, and adjacent groups among the groups represented by R¹ or R² may be bonded to each other to form a cyclic structure.

The present invention also provides a material for organic electroluminescence devices which comprises an aromatic amine derivative represented by following general formula (5): 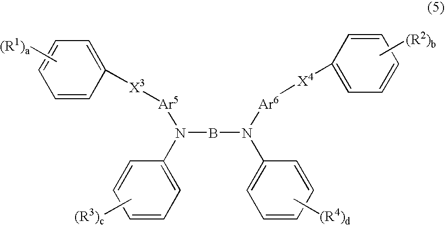
wherein

B represents a substituted or unsubstituted aromatic hydrocarbon cyclic group having 10 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;

Ar⁵ and Ar⁶ each independently represent a substituted or unsubstituted divalent aromatic hydrocarbon cyclic group having 6 to 40 nuclear carbon atoms;

X³ and X⁴ each independently represent a group represented by following formula:

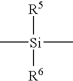

R⁵ and R⁶ each independently representing hydrogen atom, a substituted or unsubstituted alkyl group having 1 to 10 carbon atoms or a substituted or unsubstituted aryl group having 5 to 20 nuclear carbon atoms;

R¹ to R⁴ each independently represent hydrogen atom, a substituted or unsubstituted alkyl group having 1 to 50 carbon atoms, a substituted or unsubstituted aryl group having 5 to 50 nuclear carbon atoms, a substituted or unsubstituted aralkyl group having 6 to 50 nuclear carbon atoms, a substituted or unsubstituted cycloalkyl group having 3 to 50 nuclear carbon atoms, a substituted or unsubstituted alkoxyl group having 1 to 50 carbon atoms, a substituted or unsubstituted aryloxyl group having 5 to 50 nuclear carbon atoms, a substituted or unsubstituted arylamino group having 5 to 50 nuclear carbon atoms, a substituted or unsubstituted alkylamino group having 1 to 20 carbon atoms or a substituted or unsubstituted heterocyclic group having 5 to 50 nuclear carbon atoms; and

a to d each represent an integer of 1 to 5 and, when any of a to d represents an integer of 2 or greater, groups in ( ) may be same with or different from each other, and adjacent groups among the groups represented by R¹ to R⁴ may be bonded to each other to form a cyclic structure.

The present invention also provides a material for organic electroluminescence devices which comprises an aromatic amine derivative represented by following general formula (6): 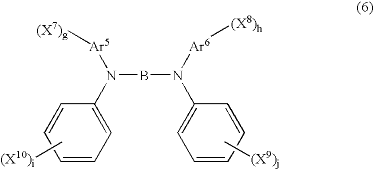
wherein

B represents a substituted or unsubstituted aromatic hydrocarbon cyclic group having 10 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;

Ar⁵ and Ar⁶ each independently represent a substituted or unsubstituted divalent aromatic hydrocarbon cyclic group having 6 to 40 nuclear carbon atoms;

X⁷ to X¹⁰ each independently represent a group represented by following formula:

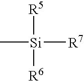

R⁵ to R⁷ each independently representing hydrogen atom, a substituted or unsubstituted alkyl group having 1 to 10 carbon atoms or a substituted or unsubstituted aryl group having 5 to 20 nuclear carbon atoms; and

g, h, i and j each represent an integer of 0 or 1, and a case where all of g to j represent 0 is excluded.

The present invention also provides an organic electroluminescence device comprising an anode, a cathode and an organic thin film layer which comprises a single layer or a plurality of layers comprising at least a light emitting layer and is disposed between the anode and the cathode, wherein at least one layer in the organic thin film layer comprises the material for organic electroluminescence devices described above singly or as a component of a mixture.

### THE EFFECT OF THE INVENTION

The organic EL device using the material for organic EL devices of the present invention provides a sufficient luminance of emitted light under application of a low voltage and a great efficiency of light emission, suppresses degradation and has a long life.

### BRIEF DESCRIPTION OF THE DRAWINGS

FIG. 1 shows a diagram exhibiting the ¹H-NMR spectrum of the aromatic amine derivative obtained in Preparation Example 1.

FIG. 2 shows a diagram exhibiting the ¹H-NMR spectrum of the aromatic amine derivative obtained in Preparation Example 3.

FIG. 3 shows a diagram exhibiting the wavelength of the maximum fluorescence of the aromatic amine derivative obtained in Preparation Example 3.

### THE MOST PREFERRED EMBODIMENT TO CARRY OUT THE INVENTION

The material for organic electroluminescence devices comprises an aromatic amine derivative represented by the following general formula (1) or general formula (2) and preferably by the following general formula (3) or general formula (4).

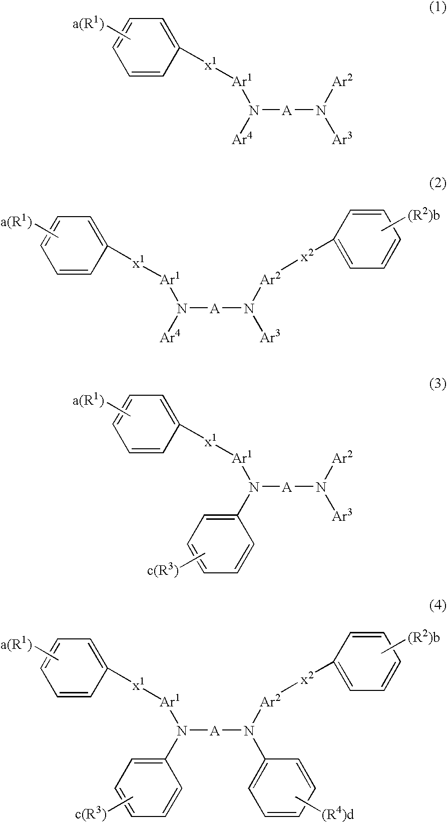

In general formulae (1) to (4), A represents a substituted or unsubstituted aromatic hydrocarbon cyclic group having 6 to 40 nuclear carbon atoms, a substituted or unsubstituted aromatic heterocyclic group having 5 to 40 nuclear carbon atoms or a divalent group comprising 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms.

Examples of the group represented by A include divalent residue groups derived from aromatic hydrocarbon cyclic compounds such as benzene, toluene, xylene, ethylbenzene, naphthalene, anthracene, phenanthrene, fluorene, pyrene, chrysene, naphthacene, perylene, azulene, fluorenone, indenofluorene, anthraquinone, dibenzosuberenone and tetracyanoquinodimethane; divalent residue groups derived from aromatic heterocyclic compounds such as furan, thiophene, pyrrol, pyridine, oxazole, pyrazine, oxadiazole, triazole, thiadiazole, indole, quinoline, isoquinoline, carbazole, acridine, thioxanthone, coumarine, acridone, diphenylene sulfone, quinoxaline, benzothiazole, phenazine, phenanthroline, phenothiazine, quinacridone, flavanthrone and indanthrone; and divalent residue groups comprising 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms, such as divalent residue groups derived from biphenyl, terphenyl, binaphthyl, bifluorenylidene, bipyridine, biquinoline, flavone, phenyltriazine, bisbenzothiazole, bithiophene, phenylbenzotriazole, phenylbenzimidazole, phenylacridine, bis-(benzoxazolyl)thiophene, bis(phenyloxazolyl)benzene, biphenylylphenyloxadiazole, diphenylbenzoquinone, diphenylisobenzofuran, diphenylpyridine, stilbene, dibenzyl, diphenylmethane, bis(phenylisopropyl)-benzene, diphenylfluorene, diphenylhexafluoropropane, dibenzyl naphthyl ketone, (phenylethyl)benzylnaphthalene, diphenyl ether, methyl-diphenylamine, benzophenone, phenyl benzoate, diphenylurea, diphenyl sulfide, diphenyl sulfone, diphenoxybiphenyl, bis(phenoxyphenyl) sulfone, bis(phenoxyphenyl)propane, diphenoxybenzene and dipyridylamine.

Among these groups, divalent residue groups derived from naphthalene, anthracene, phenanthrene, fluorene, pyrene, chrysene, naphthacene and perylene are preferable.

Typical examples of the group represented by A are shown in the following. However, the group represented by A is not limited to the groups shown as the examples.

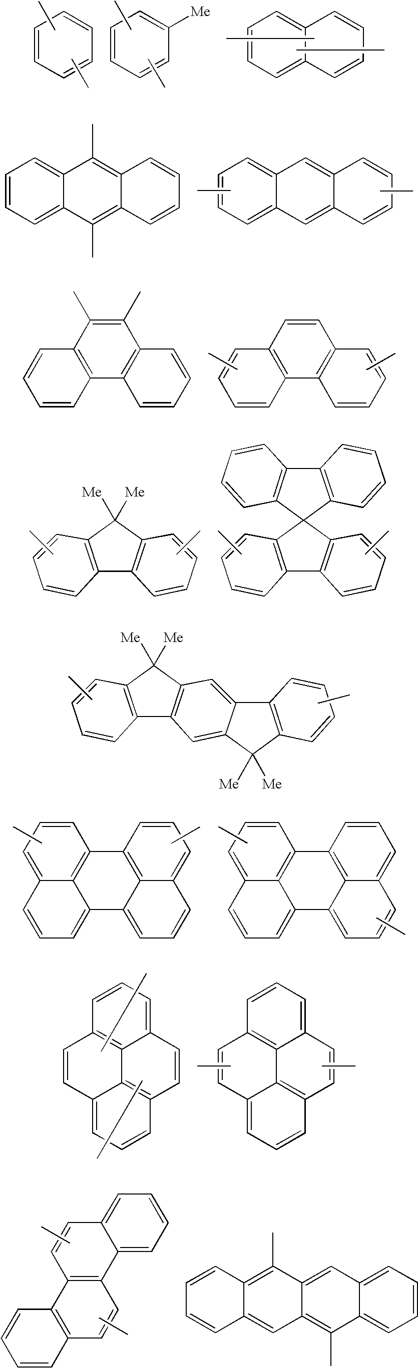

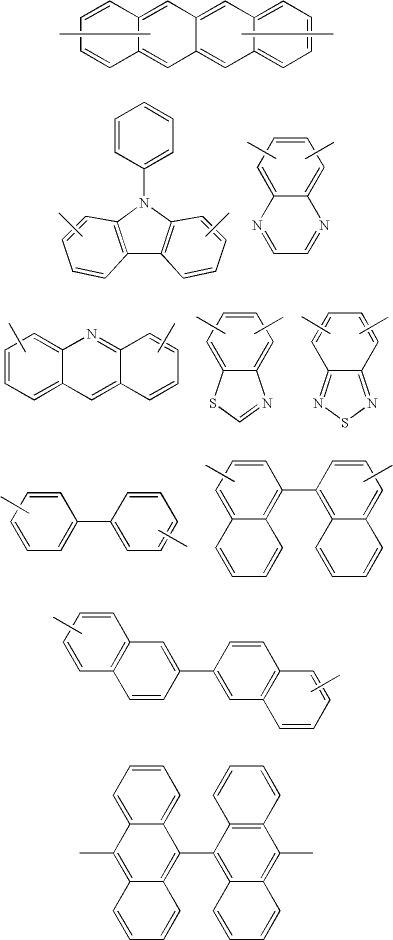

In general formulae (1) to (4), Ar¹ to Ar⁴ each independently represent a substituted or unsubstituted aromatic hydrocarbon cyclic group having 6 to 40 nuclear carbon atoms or a substituted or unsubstituted aromatic heterocyclic group having 5 to 40 nuclear carbon atoms, wherein Ar¹ represents a divalent group, Ar² represents a monovalent or a divalent group and Ar³ and Ar⁴ each represent a monovalent group.

Examples of the group represented by Ar¹ to Ar⁴ include monovalent or divalent residue groups derived from benzene, toluene, xylene, ethylbenzene, naphthalene, anthracene, phenanthrene, fluorene, pyrene, chrysene, naphthacene, perylene and azulene.

In general formulae (1) to (4), X¹ to X⁴ each independently represent —O—, —S—, >C═O, >SO₂, —(CₓH₂ₓ)—O—(C_{y}H_{2y})—, x and y each representing an integer of 0 to 20 excluding a case where x+y=0, a substituted or unsubstituted alkylidene group having 2 to 20 carbon atoms, a substituted or unsubstituted alkylene group having 2 to 20 carbon atoms or a substituted or unsubstituted divalent aliphatic cyclic group having 3 to 10 nuclear carbon atoms.

Examples of the alkylidene group include propylidene group, isopropylidene group, butylidene group and pentylidene group.

Examples of the aliphatic cyclic group include divalent groups derived from cyclopentyl group, cyclohexyl group, 4-methylcyclohexyl group and cycloheptyl group.

Examples of the alkylene group include divalent groups derived from the alkyl groups represented by R¹ to R⁴ described in the following.

As the atom and the group represented by X¹ to X⁴, oxygen atom, sulfur atom, methylene group, isopropylene group, cyclohexylene group, phenylene group, carbonyl group and diphenylmethylene group are preferable among the above atoms and groups.

In general formulae (1), to (4), R¹ to R⁴ each independently represent hydrogen atom, a substituted or unsubstituted alkyl group having 1 to 50 carbon atoms and preferably 1 to 20 carbon atoms, a substituted or unsubstituted aryl group having 5 to 50 nuclear carbon atoms and preferably 5 to 20 nuclear carbon atoms, a substituted or unsubstituted aralkyl group having 6 to 50 nuclear carbon atoms and preferably 6 to 20 nuclear carbon atoms, a substituted or unsubstituted cycloalkyl group having 3 to 50 nuclear carbon atoms and preferably 5 to 12 nuclear carbon atoms, a substituted or unsubstituted alkoxyl group having 1 to 50 carbon atoms and preferably 1 to 6 carbon atoms, a substituted or unsubstituted aryloxyl group having 5 to 50 nuclear carbon atoms and preferably 5 to 18 nuclear carbon atoms, a substituted or unsubstituted arylamino group having 5 to 50 nuclear carbon atoms and preferably 5 to 18 nuclear carbon atoms, a substituted or unsubstituted alkylamino group having 1 to 20 carbon atoms and preferably 1 to 6 carbon atoms or a substituted or unsubstituted heterocyclic group having 5 to 50 nuclear carbon atoms 5 to 20 nuclear carbon atoms.

Examples of the alkyl group represented by R¹ to R⁴ include methyl group, ethyl group, propyl group, isopropyl group, butyl group, s-butyl group, t-butyl group, pentyl group, hexyl group, heptyl group, octyl group, stearyl group, trichloromethyl group and trifluoromethyl group.

Examples of the aryl group represented by R¹ to R⁴ include phenyl group, 2-methylphenyl group, 3-methylphenyl group, 4-methylphenyl group, 4-ethylphenyl group biphenyl group, 4-methylbiphenyl group, 4-ethylbiphenyl group, 4-cyclohexylbiphenyl group, terphenyl group, 3,5-dichlorophenyl group, naphthyl group, 5-methylnaphthyl group, anthryl group and pyrenyl group.

Examples of the cycloalkyl group represented by R¹ to R⁴ include cyclopropyl group, cyclobutyl group, cyclopentyl group and cyclohexyl group.

Examples of the aralkyl group represented by R¹ to R⁴ include benzyl group, α,α-methylphenylbenzyl group, triphenylmethyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, 2-β-naphthylisopropyl group, α-phenoxybenzyl group, α-benzyloxy-benzyl group, α,α-ditrifluoromethylbenzyl group, 1-pyrrolylmethyl group, 2-(1-pyrrolyl)ethyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group and 1-chloro-2-phenylisopropyl group.

Examples of the heterocyclic group represented by R¹ to R⁴ include pyridinyl group, pyrazinyl group, pyrimidinyl group, pyridazinyl group, triazinyl group, indolinyl group, quinolinyl group, acridinyl group, pyrrolidinyl group, dioxanyl group, piperidinyl group, morpholidinyl group, piperazinyl group, triatinyl group, carbazolyl group, furanyl group, thiophenyl group, oxazolyl group, oxadiazolyl group, benzoxazolyl group, thiazolyl group, thiadiazolyl group, benzothiazolyl group, triazolyl group, imidazolyl group, benzimidazolyl group and planyl group.

Examples of the alkoxyl group represented by R¹ to R⁴ include methoxyl group, ethoxyl group, propoxyl group, isopropoxyl group, butoxyl group, isobutoxyl group, s-butoxyl group, t-butoxyl group, various types of pentyloxyl groups and various types of hexyloxyl groups.

Examples of the aryloxyl group represented by R¹ to R⁴ include phenoxyl group, tolyloxyl group and naphthyloxyl group.

Examples of the arylamino group represented by R¹ to R⁴ include diphenylamino group, ditolylamino group, dinaphthylamino group and naphthylphenylamino group.

Examples of the alkylamino group represented by R¹ to R⁴ include dimethylamino group, diethylamino group and dihexylamino group.

In general formulae (1) to (4), a to d each represent an integer of 1 to 5 and, when any of a to d represents an integer of 2 or greater, groups in ( ) may be same with or different from each other and adjacent groups among groups represented by R¹ to R⁴ may be bonded to each other to form a cyclic structure.

Examples of the cyclic structure which may be formed with groups adjacent to each other include cycloheptene ring, cyclohexene ring, phenyl ring, naphthalene ring, anthracene ring, pyrene ring, fluorene ring, furan ring, thiophene ring, pyrrol ring, oxazole ring, thiazole ring, imidazole ring, pyridine ring, pyrazine ring, pyrroline ring, pyrazoline ring, indole ring, quinoline ring, quinoxaline ring, xanthenes ring, carbazole ring, acridine ring and phenanthroline ring, which are substituted or unsubstituted.

As for the aromatic amine derivatives represented by general formulae (3) and (4), it is preferable that R³ represents a secondary or tertiary alkyl group, and it is more preferable that c represents an integer of 2 or 3 in general formulae (3) and (4).

Examples of the substituent to the groups in the compounds represented by general formulae (1) to (4) include alkyl groups (such as methyl group, ethyl group, propyl group, isopropyl group, n-butyl group, s-butyl group, isobutyl group, t-butyl group, n-pentyl group, n-hexyl group, n-heptyl group, n-octyl group, hydroxymethyl group, 1-hydroxyethyl group, 2-hydroxyethyl group, 2-hydroxyisobutyl group, 1,2-dihydroxyethyl group, 1,3-dihydroxy-isopropyl group, 2,3-dihydroxy-t-butyl group, 1,2,3-trihydroxypropyl group, chloromethyl group, 1-chloroethyl group, 2-chloroethyl group, 2-chloroisobutyl group, 1,2-dichloroethyl group, 1,3-dichloroisopropyl group, 2,3-dichloro-t-butyl group, 1,2,3-trichloropropyl group, bromo-methyl group, 1-bromoethyl group, 2-bromoethyl group, 2-bromoisobutyl group, 1,2-dibromoethyl group, 1,3-dibromoisopropyl group, 2,3-dibromo-t-butyl group, 1,2,3-tribromopropyl group, iodomethyl group, 1-iodoethyl group, 2-iodoethyl group, 2-iodoisobutyl group, 1,2-diiodoethyl group, 1,3-diiodoisopropyl group, 2,3-diiodo-t-butyl group, 1,2,3-triiodopropyl group, aminomethyl group, 1-aminoethyl group, 2-aminoethyl group, 2-aminoisobutyl group, 1,2-diaminoethyl group, 1,3-diaminoisopropyl group, 2,3-diamino-t-butyl group, 1,2,3-triaminopropyl group, cyanomethyl group, 1-cyanoethyl group, 2-cyanoethyl group, 2-cyanoisobutyl group, 1,2-dicyanoethyl group, 1,3-dicyanoisopropyl group, 2,3-dicyano-t-butyl group, 1,2,3-tricyanopropyl group, nitromethyl group, 1-nitroethyl group, 2-nitroethyl group, 2-nitroisobutyl group, 1,2-dinitroethyl group, 1,3-dinitroisopropyl group, 2,3-dinitro-t-butyl group, 1,2,3-trinitropropyl group, cyclopropyl group, cyclobutyl group, cyclopentyl group, cyclohexyl group, 4-methylcyclohexyl group, 1-adamantyl group, 2-adamantyl group, 1-norbornyl group and 2-norbornyl group), alkoxyl groups having 1 to 6 carbon atoms (such as ethoxyl group, methoxyl group, i-propoxyl group, n-propoxyl group, s-butoxyl group, t-butoxyl group, pentoxyl group, hexyloxyl group, cyclopentoxyl group and cyclohexyloxyl group), aryl groups having 5 to 40 nuclear atoms, amino groups substituted with aryl groups having 5 to 40 nuclear atoms, ester groups having aryl groups having 5 to 40 nuclear atoms, ester groups having alkyl groups having 1 to 6 carbon atoms, cyano group, nitro group and halogen atoms.

Typical examples of the group at the outside of the nitrogen atom [the portion of the substituted or unsubstituted benzene group —Xⁿ—Arⁿ— (n=1˜4)] in general formulae (1) to (4) are shown in the following. However, the above group is not limited to the group shown as the examples.

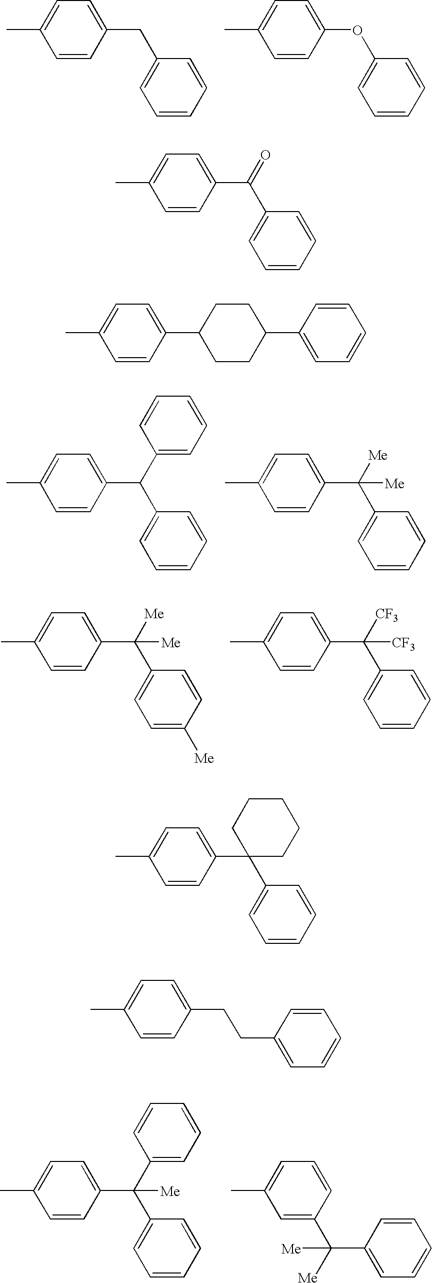

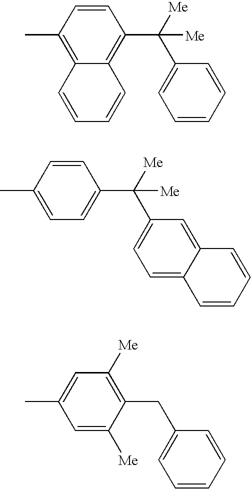

The material for organic electroluminescence devices also comprises an aromatic amine derivative represented by the following general formula (5):

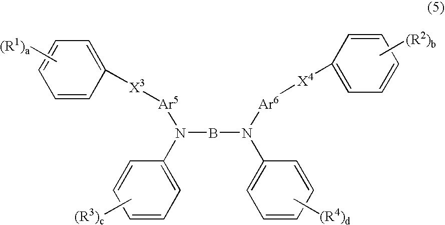

In general formula (5), B represents a substituted or unsubstituted aromatic hydrocarbon cyclic group having 10 to 40 nuclear carbon atoms, a substituted or unsubstituted aromatic heterocyclic group having 5 to 40 nuclear carbon atoms or a divalent group comprising 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms.

Examples of the group represented by B include divalent residue groups derived from aromatic hydrocarbon cyclic compounds such as naphthalene, anthracene, phenanthrene, fluorene, pyrene, chrysene, naphthacene, perylene, azulene, fluorenone, indenofluorene, anthraquinone, dibenzosuberenone and tetracyanoquinodimethane; divalent residue groups derived from aromatic heterocyclic compounds such as furan, thiophene, pyrrol, pyridine, oxazole, pyrazine, oxadiazole, triazole, thiadiazole, indole, quinoline, isoquinoline, carbazole, acridine, thioxanthone, coumarine, acridone, diphenylene sulfone, quinoxaline, benzothiazole, phenazine, phenanthroline, phenothiazine, quinacridone, flavanthrone and indanthrone; and divalent residue groups comprising 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms, such as divalent residue groups derived from biphenyl, terphenyl, binaphthyl, bifluorenylidene, bipyridine, biquinoline, flavone, phenyltriazine, bisbenzothiazole, bithiophene, phenylbenzotriazole, phenylbenzimidazole, phenylacridine, bis(benzoxazolyl)thiophene, bis(phenyloxazolyl)benzene, biphenylylphenyloxadiazole, diphenylbenzoquinone, diphenylisobenzofuran, diphenylpyridine, stilbene, dibenzyl, diphenylmethane, bis(phenylisopropyl)benzene, diphenylfluorene, diphenylhexafluoropropane, dibenzyl naphthyl ketone, (phenylethyl)-benzylnaphthalene, diphenyl ether, methyldiphenylamine, benzophenone, phenyl benzoate, diphenylurea, diphenyl sulfide, diphenyl sulfone, diphenoxybiphenyl, bis(phenoxyphenyl) sulfone, bis(phenoxyphenyl)-propane, diphenoxybenzene and dipyridylamine.

Among these groups, divalent residue groups derived from naphthalene, anthracene, phenanthrene, fluorene, pyrene, chrysene, naphthacene and perylene are preferable.

Typical examples of the group represented by B are shown in the following. However, the group represented by B is not limited to the groups shown as the examples.

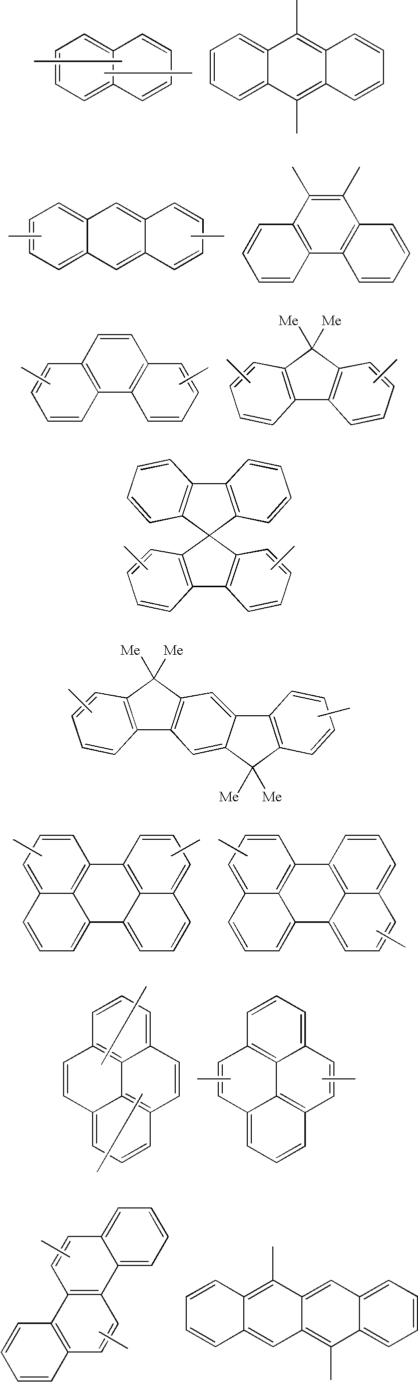

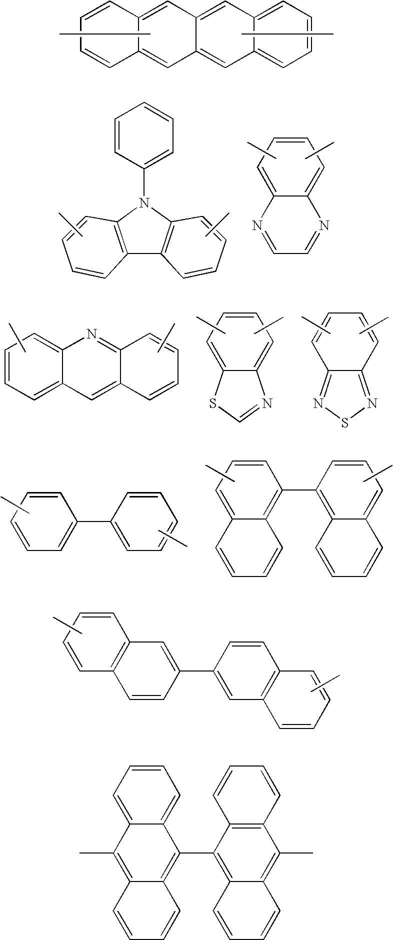

In general formula (5), Ar⁵ and Ar⁶ each independently represent a substituted or unsubstituted divalent aromatic hydrocarbon cyclic group having 6 to 40 nuclear carbon atoms. Examples of the above group include divalent residue groups derived from benzene, toluene, xylene, ethylbenzene, naphthalene, anthracene, phenanthrene, fluorene, pyrene, chrysene, naphthacene, perylene and azulene.

In general formula (5), X³ and X⁴ each independently represent a group represented by following formula: 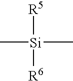
wherein R⁵ and R⁶ each independently representing hydrogen atom, a substituted or unsubstituted alkyl group having 1 to 10 carbon atoms or a substituted or unsubstituted aryl group having 5 to 20 nuclear carbon atoms. Examples and preferable groups include groups having carbon atoms in the number described above among the groups described above as the examples and the preferable groups, respectively, of the corresponding groups represented by R¹ to R⁴.

In general formula (5), a to d each represent an integer of 1 to 5 and, when any of a to d represents an integer of 2 or greater, groups in ( ) may be the same with or different from each other, and adjacent groups among the groups represented by R¹ to R⁴ may be bonded to each other to form a cyclic structure. Examples of the cyclic structure which may be formed with adjacent groups include the corresponding structures described above for general formulae (1) to (4).

The material for organic electroluminescence devices of the present invention comprises an aromatic amine derivative represented by following general formula (6):

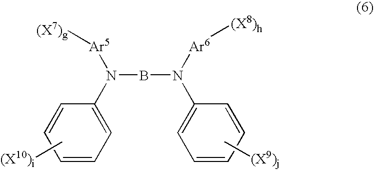

In general formula (6), B and Ar⁵ to Ar⁶ are as defined in general formula (5), and examples, preferable groups and preferable substituents are as described for general formula (5).

In general formula (6), it is preferable that B represents a divalent residue group derived from substituted or unsubstituted naphthalene, anthracene, pyrene or chrysene.

In general formula (6), X⁷ to X¹⁰ each independently represent a group represented by following formula: 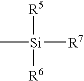
wherein R⁵ to R⁷ each independently representing hydrogen atom, a substituted or unsubstituted alkyl group having 1 to 10 carbon atoms or a substituted or unsubstituted aryl group having 5 to 20 nuclear carbon atoms. Examples of these groups include groups having carbon atoms in the number described above among the groups described above as the examples of the corresponding groups represented by R¹ to R⁴.

In general formula (6), g, h, i and j each represent an integer of 0 or 1, and the case where all of g to j represent 0 is excluded. It is preferable that g, h, i and j all represent 1.

Since the aromatic amine derivative used as the material for organic EL devices in the present invention has a bulky group having a great molecular weight, the glass transition temperature and the melting point are high. The compounds represented by general formula (1) to (5) in which the adjacent groups represented by R¹ to R⁴ form a cyclic structure have still higher glass transition temperatures and melting points. Therefore, resistance to Joule heat generated in an organic layer, at the interface of organic layers and at the interface of an organic layer and a metal electrode (heat resistance) during light emission under the electric field is improved. When the compound is used as the light emitting material for an organic EL device, a great luminance of emitted light is obtained, and the device is advantageous for emitting light for a long period of time.

Examples of the aromatic amine derivatives represented by general formulae (1) to (6) of the present invention are shown in the following. However, the aromatic amine derivative is not limited to the compounds shown as the examples. Me represents methyl group.

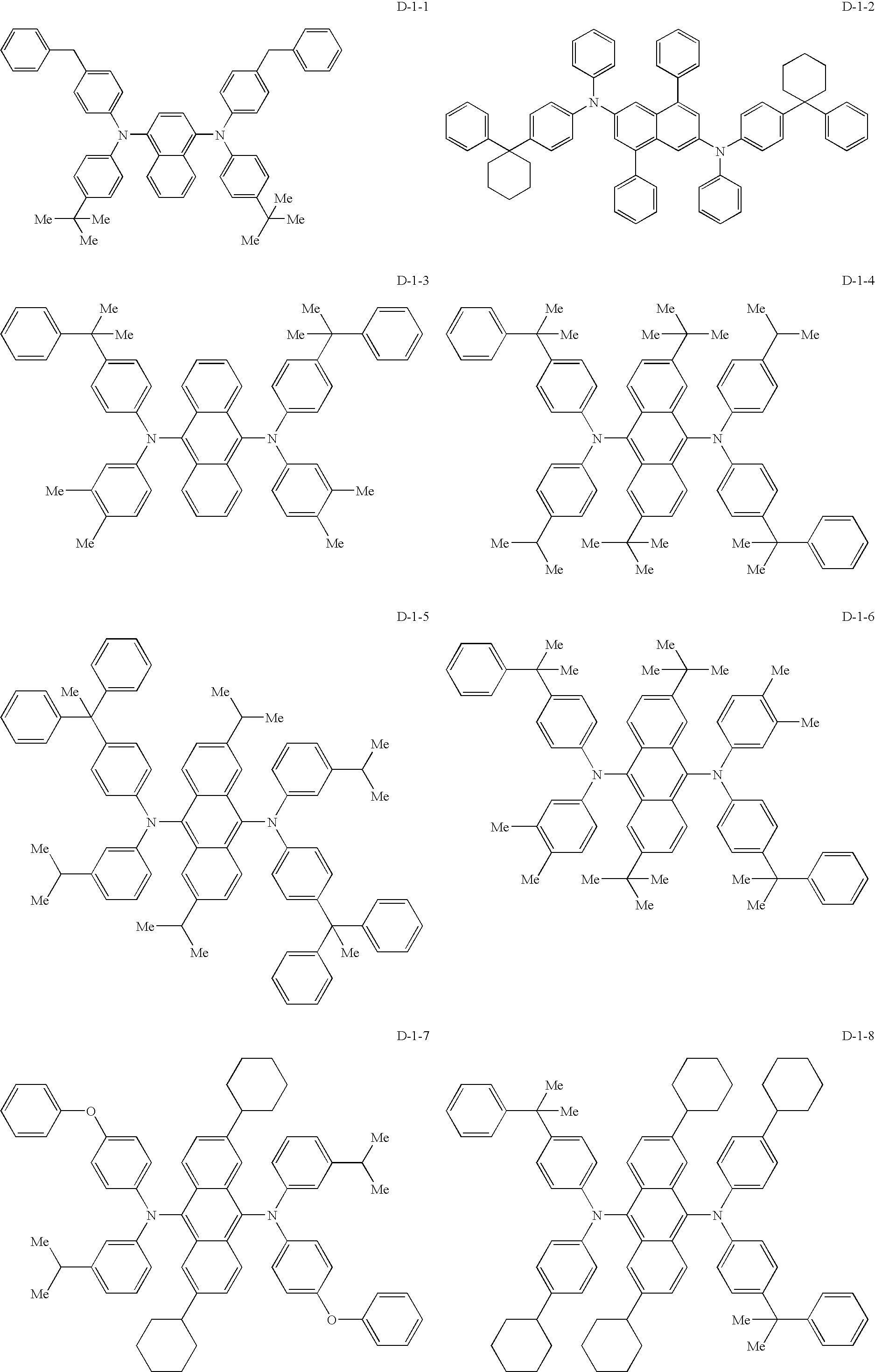

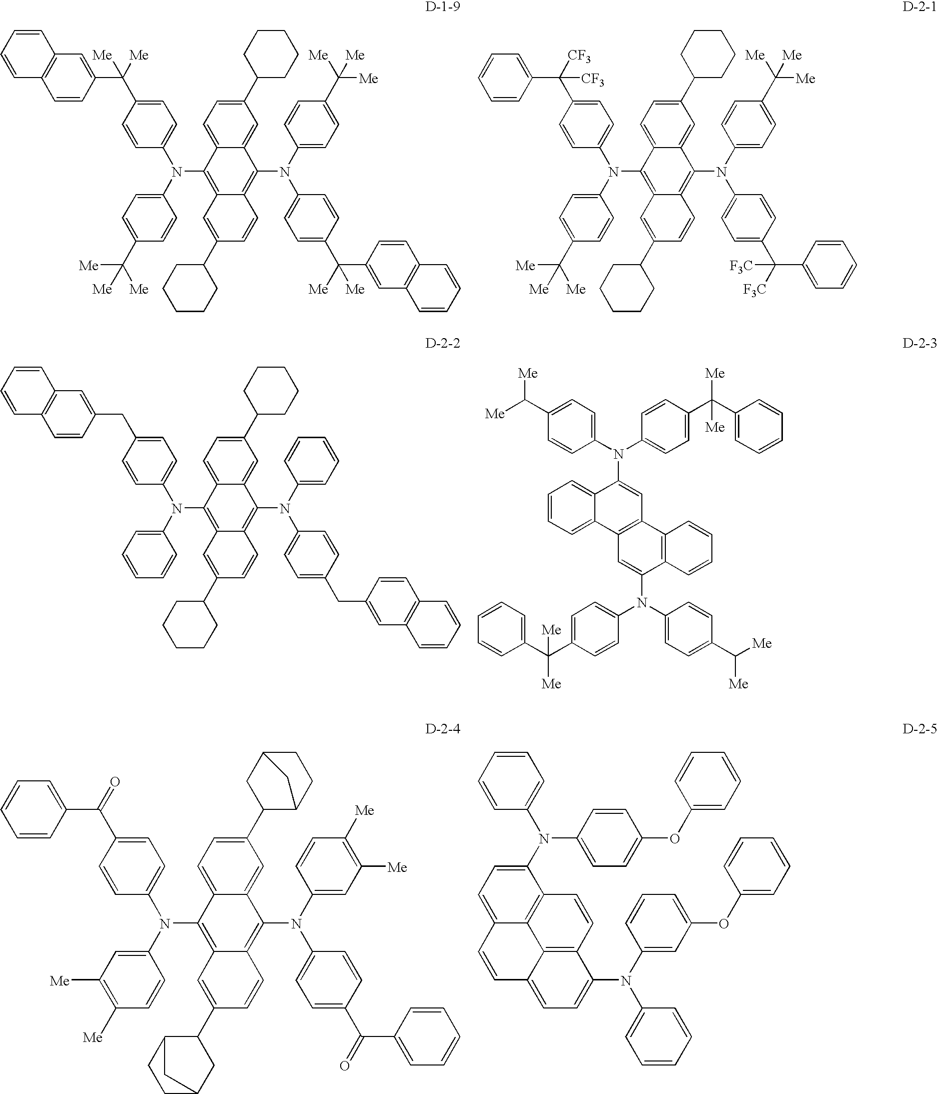

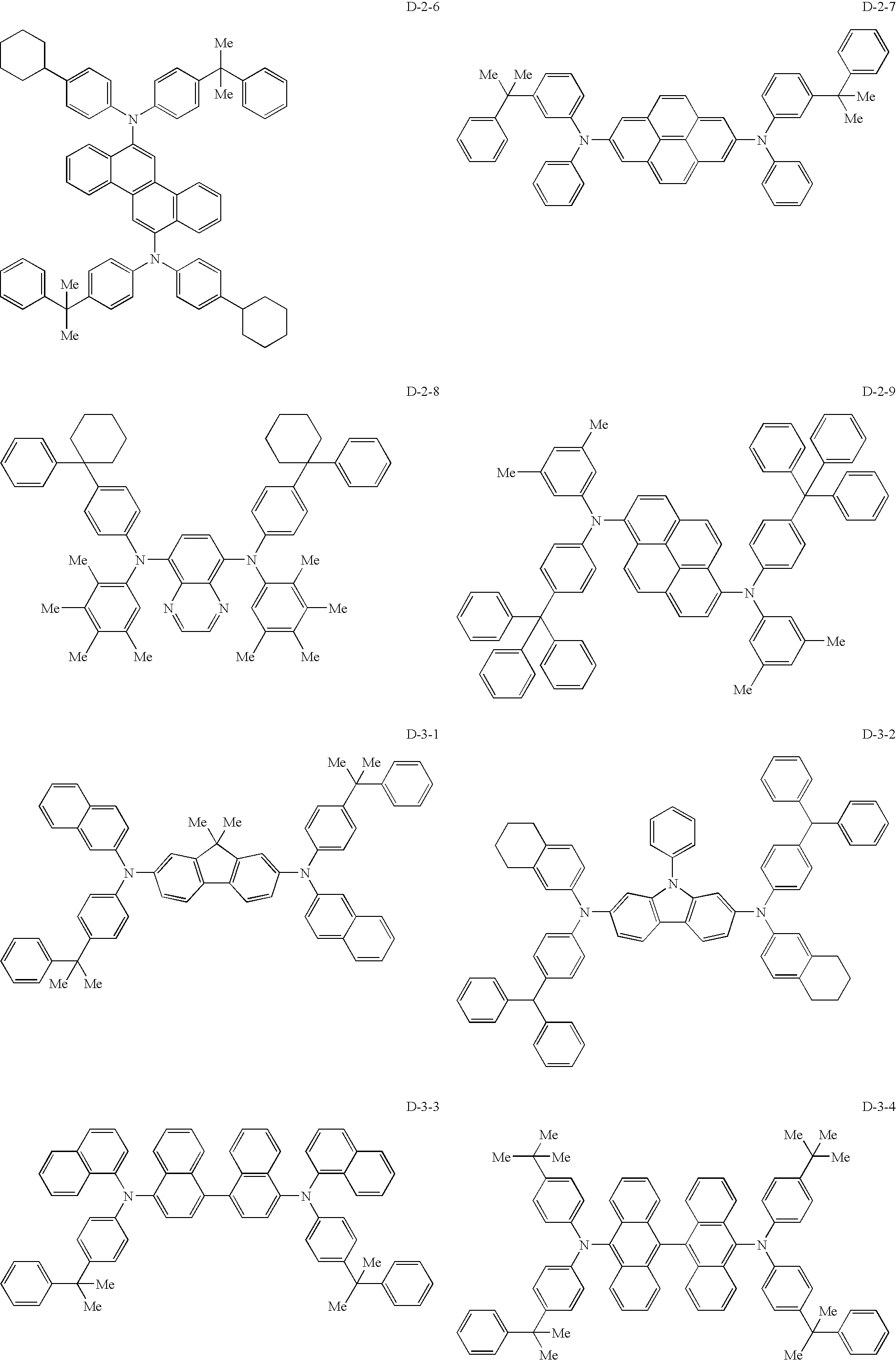

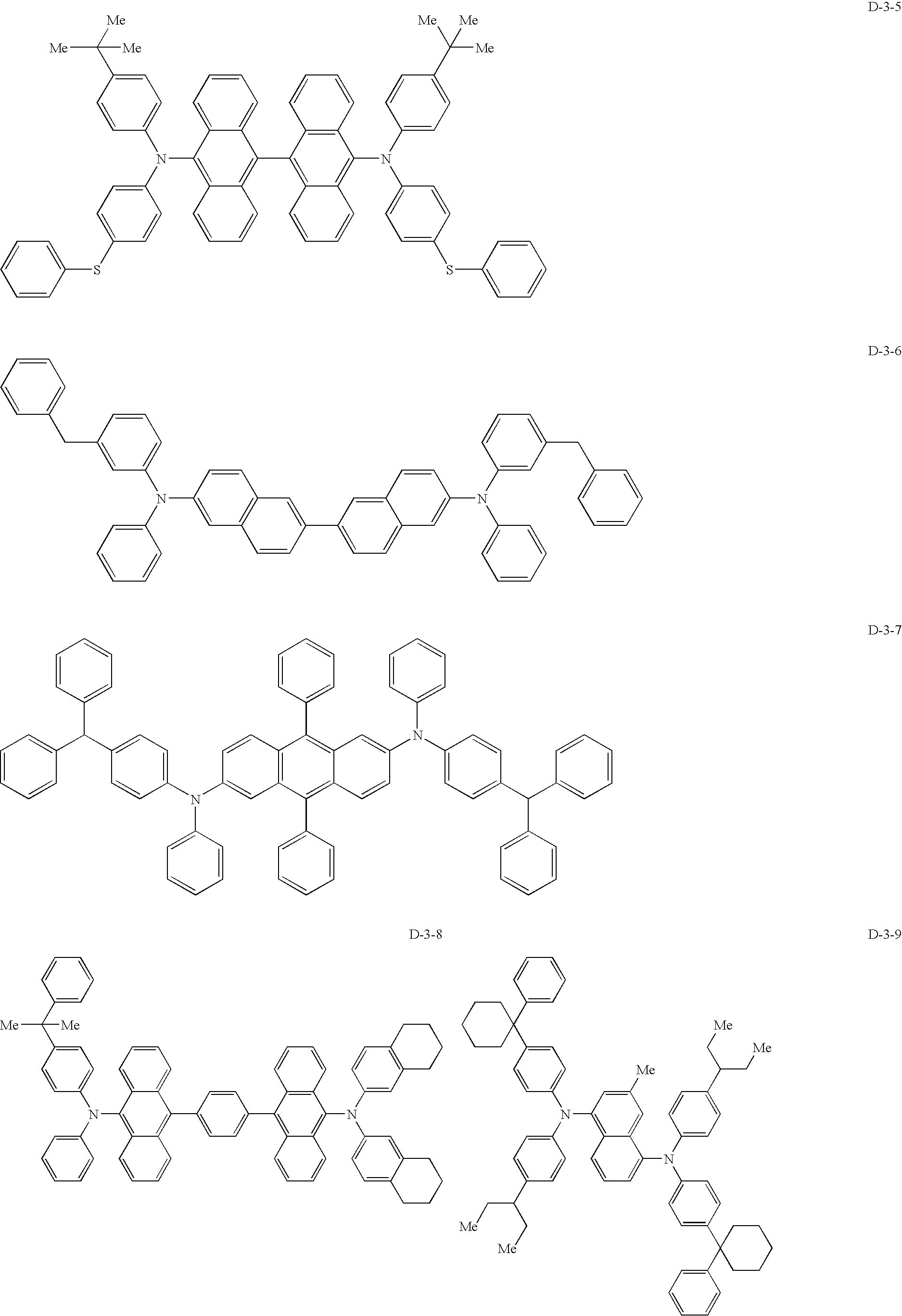

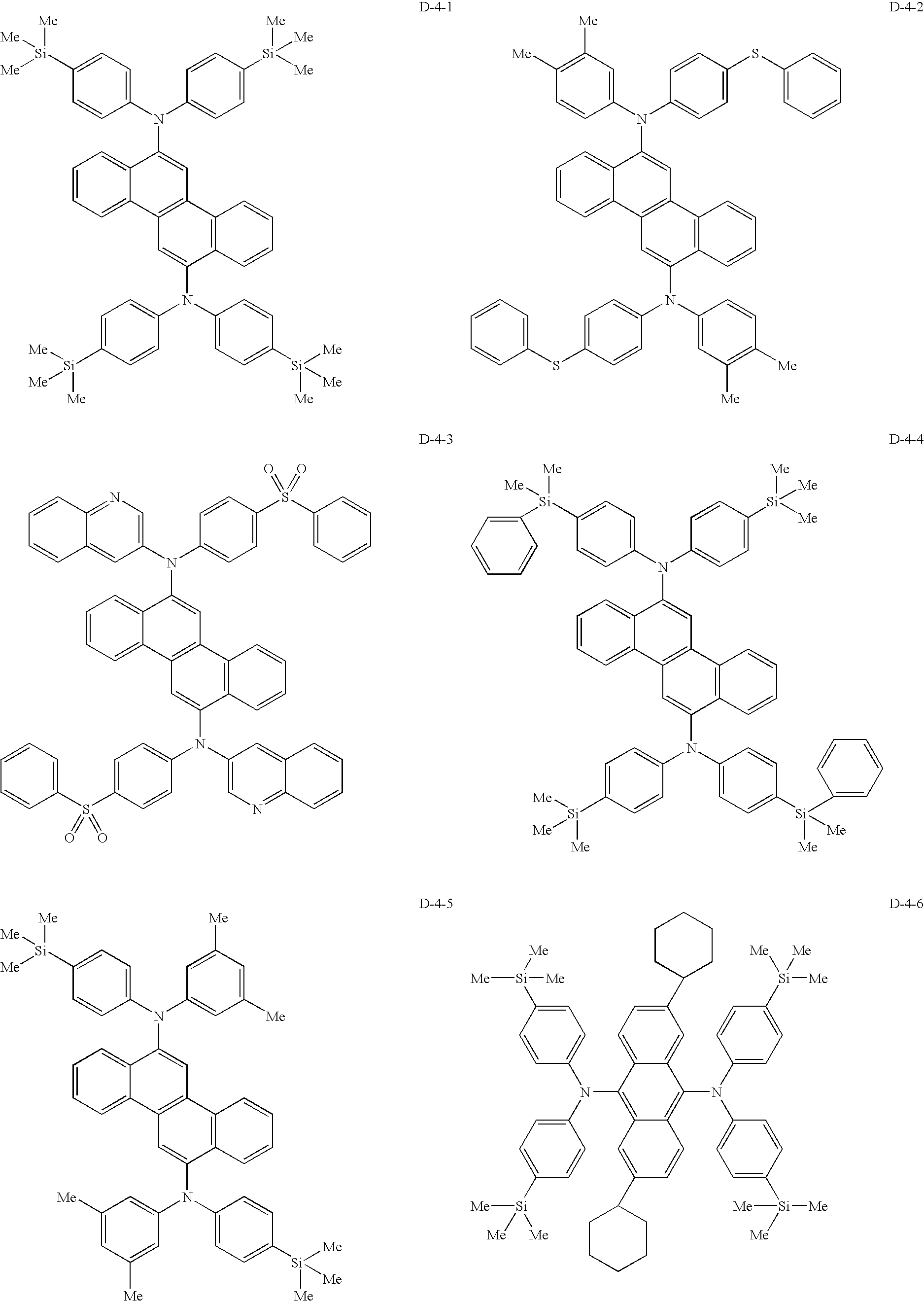

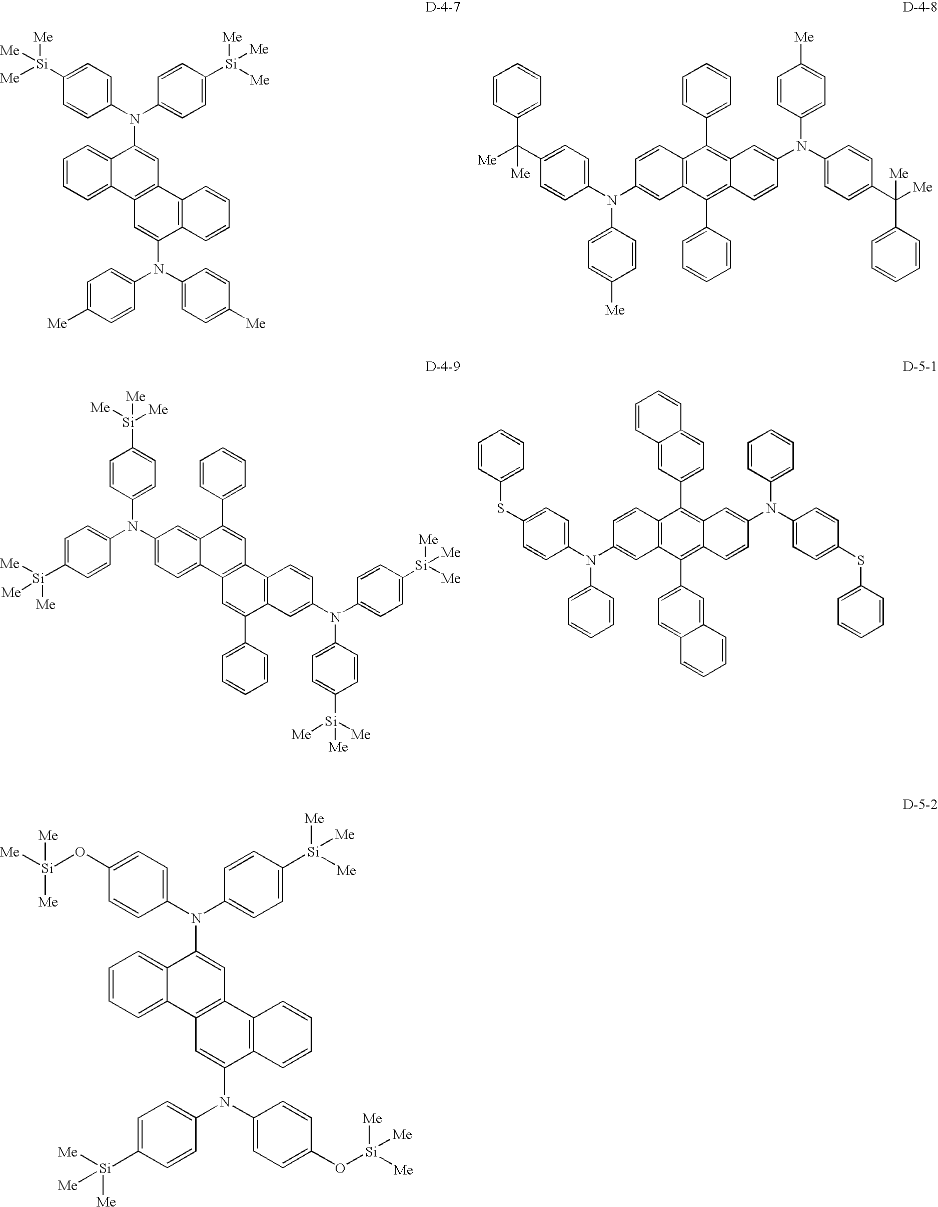

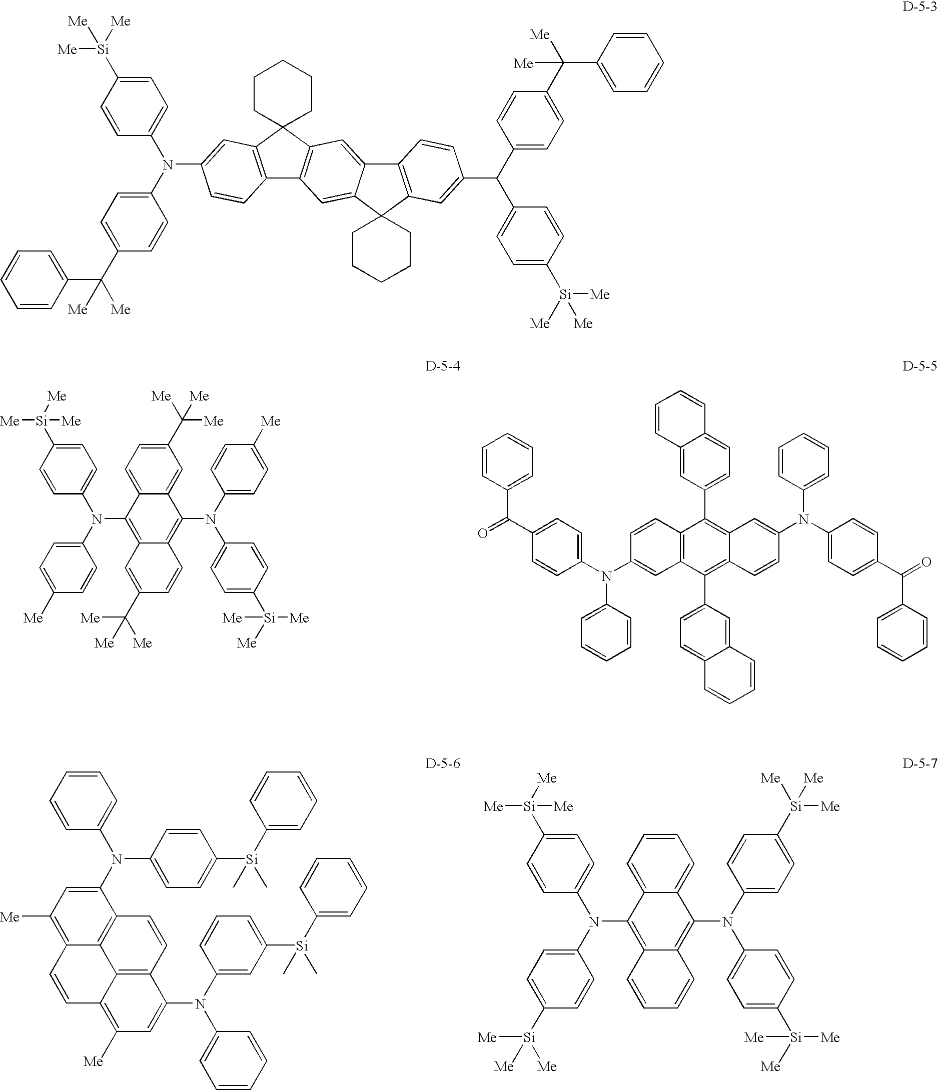

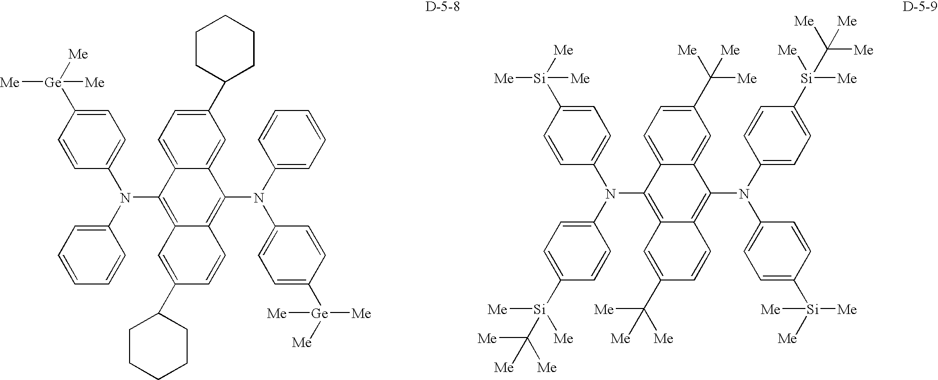

The organic EL device comprising the aromatic the aromatic amine derivative represented by one of general formulae (1) to (6) of the present invention has a long life since the steric repulsion between the condensed polycyclic hydrocarbon structure as the center of light emission and the amine structure is great due to the bulky substituent having the benzene ring at the end portion of the structure, and association between the compounds can be prevented.

The aromatic amine derivative of the present invention has the strong fluorescent property in the solid state, the excellent property for light emission under application of the electric field and a quantum efficiency of fluorescence of 0.3 or greater. The aromatic amine derivative further exhibits a combination of the excellent property for hole injection and hole transportation from a metal electrode or an organic thin film layer and the excellent property for electron injection and electron transportation from a metal electrode or an organic thin film layer. Therefore, the aromatic amine derivative can be effectively used as the light emitting material for organic EL device, in particular, as the doping material. The aromatic amine derivative may be used in combination with other hole injecting and transporting materials, electron injecting and transporting materials and doping materials.

The organic EL device of the present invention is a device prepared by forming at least one organic thin film layer between an anode and a cathode. When the organic thin film layer comprises a single layer, a light emitting layer is disposed between the anode and the cathode. The light emitting layer comprises a light emitting material and may further comprise a hole injecting material or an electron injecting material for transporting holes injected from the anode or electrons injected from the cathode, respectively, to the light emitting material. The aromatic amine derivative used in the present invention exhibits the excellent light emitting property, the excellent property for hole injection and hole transportation and the excellent property for electron injection and electron transportation, and the derivative can be used for the light emitting layer as the light emitting material or the doping material.

In the organic EL device of the present invention, it is preferable that the light emitting layer comprises the aromatic amine derivative of the present invention singly or as a component of a mixture. It is preferable that the content of the aromatic amine derivative is 0.1 to 20% by weight and more preferably 1 to 10% by weight. Since the aromatic amine derivative of the present invention exhibits the combination of the very great quantum efficiency of light emission, the excellent hole transporting ability and the excellent electron transporting ability and forms a uniform thin film, it is possible that the light emitting layer is formed with the aromatic amine derivative alone.

It is preferable that the organic EL device of the present invention comprises an organic layer comprising the aromatic amine derivative of the present invention disposed between the anode and the light emitting layer. Examples of the organic layer include a hole injecting layer and a hole transporting layer.

The material for organic EL devices of the present invention is advantageous as the doping material. When the material for organic EL devices is used as the doping material, it is preferable that the material comprises as the host material at least one compound selected from anthracene derivatives represented by the following general formula (7), anthracene derivatives represented by the following general formula (8) and pyrene derivatives represented by the following general formula (9):

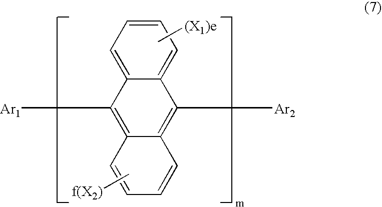

In general formula (7), X₁ and X₂ each independently represent hydrogen atom, a substituted or unsubstituted alkyl group having 1 to 50 carbon atoms, a substituted or unsubstituted aryl group having 5 to 50 carbon atoms, a substituted or unsubstituted aralkyl group having 1 to 50 carbon atoms, a substituted or unsubstituted cycloalkyl group having 5 to 50 carbon atoms, a substituted or unsubstituted alkoxyl group having 1 to 50 carbon atoms, a substituted or unsubstituted aryloxyl group having 5 to 50 carbon atoms, a substituted or unsubstituted arylamino group having 5 to 50 carbon atoms, a substituted or unsubstituted alkylamino group having 1 to 20 carbon atoms, a substituted or unsubstituted heterocyclic group having 5 to 50 carbon atoms or a halogen atom, e and f each independently represent an integer of 0 to 4 and, when any of e and f represent an integer of 2 or greater, a plurality of atoms and groups represented by X₁ and X₂ may be the same with or different from each other.

Ar₁ and Ar₂ each independently represent a substituted or unsubstituted aryl group having 5 to 50 carbon atoms or a substituted or unsubstituted heterocyclic group having 5 to 50 carbon atoms, and at least one of Ar₁ and Ar₂ represents a substituted or unsubstituted condensed ring aryl group having 10 to 50 nuclear carbon atoms or a substituted or unsubstituted aryl group having 10 or more carbon atoms.

m represents an integer of 1 to 3, and groups in [ ] may be the same with or different from each other when m represents an integer of 2 or greater.

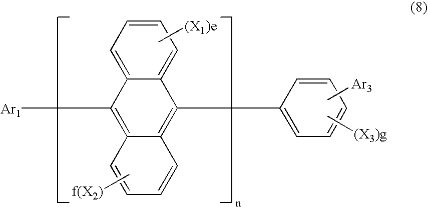

In general formula (8), X₁ and X₂ each independently represent hydrogen atom, a substituted or unsubstituted alkyl group having 1 to 50 carbon atoms, a substituted or unsubstituted aryl group having 5 to 50 carbon atoms, a substituted or unsubstituted aralkyl group having 1 to 50 carbon atoms, a substituted or unsubstituted cycloalkyl group having 5 to 50 carbon atoms, a substituted or unsubstituted alkoxyl group having 1 to 50 carbon atoms, a substituted or unsubstituted aryloxyl group having 5 to 50 carbon atoms, a substituted or unsubstituted arylamino group having 5 to 50 carbon atoms, a substituted or unsubstituted alkylamino group having 1 to 20 carbon atoms, a substituted or unsubstituted heterocyclic group having 5 to 50 carbon atoms or a halogen atom, e and f each independently represent an integer of 0 to 4, and, when any of e and f represent an integer of 2 or greater, a plurality of atoms and groups represented by X₁ and X₂ may be the same with or different from each other.

Ar₁ represents a substituted or unsubstituted condensed ring aryl group having 10 to 50 nuclear carbon atoms, and Ar₃ represents a substituted or unsubstituted aryl group having 5 to 50 nuclear carbon atoms.

n represents an integer of 1 to 3, and groups in [ ] may be the same with or different from each other when n represents an integer of 2 or greater.

Examples of the anthracene derivatives represented by general formulae (7) and (8) are shown in the following. However, the anthracene derivatives are not limited to the compounds shown as the examples.

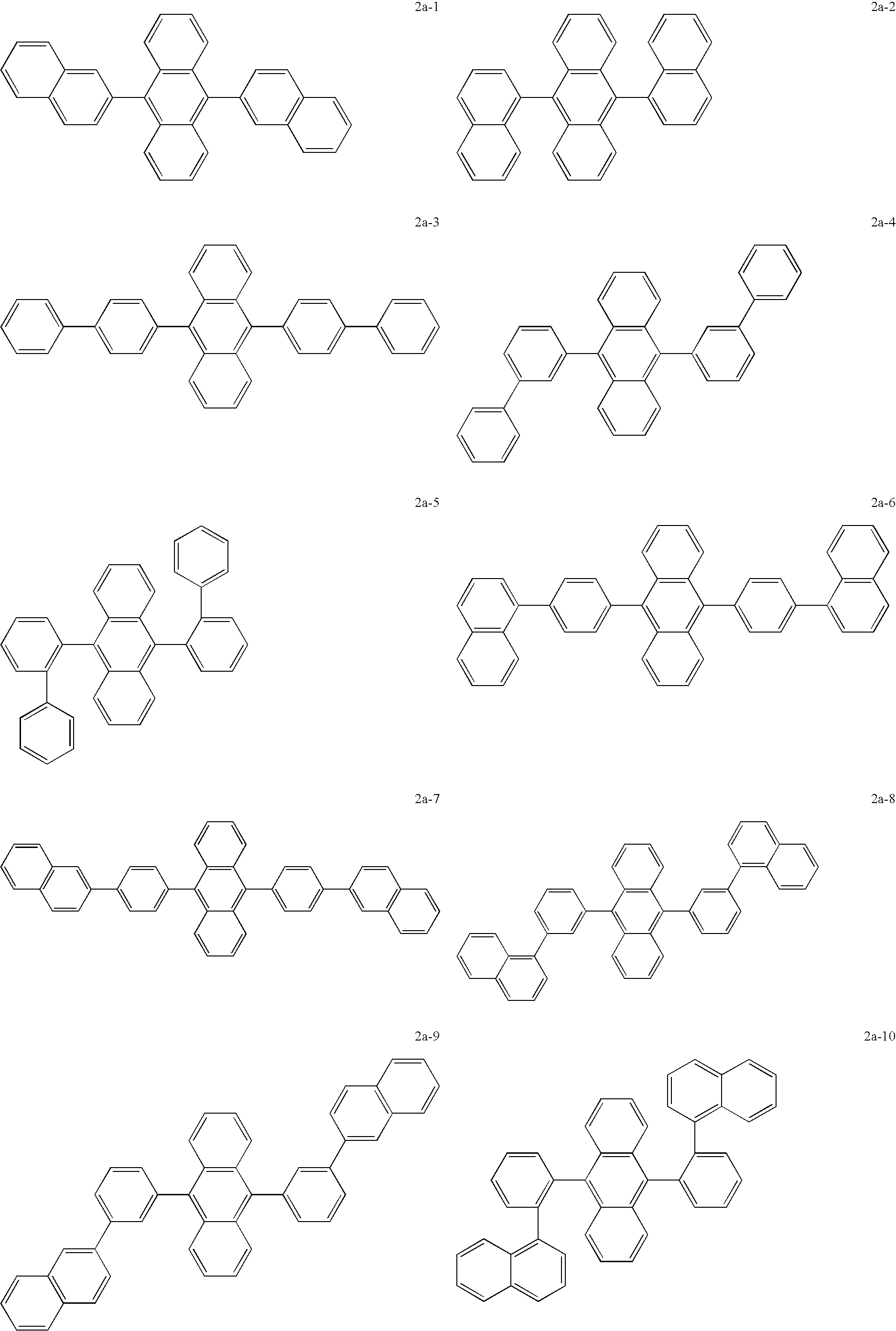

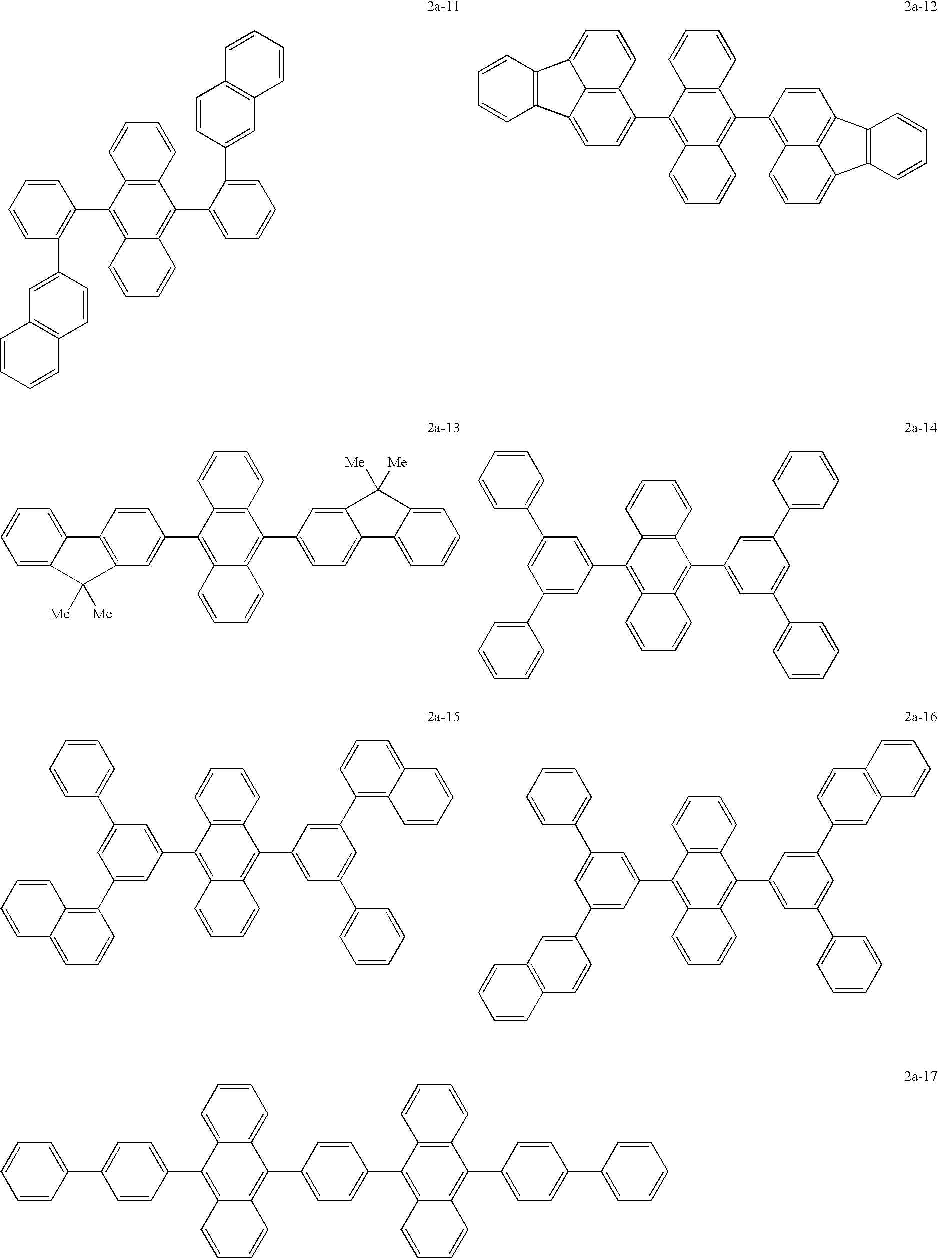

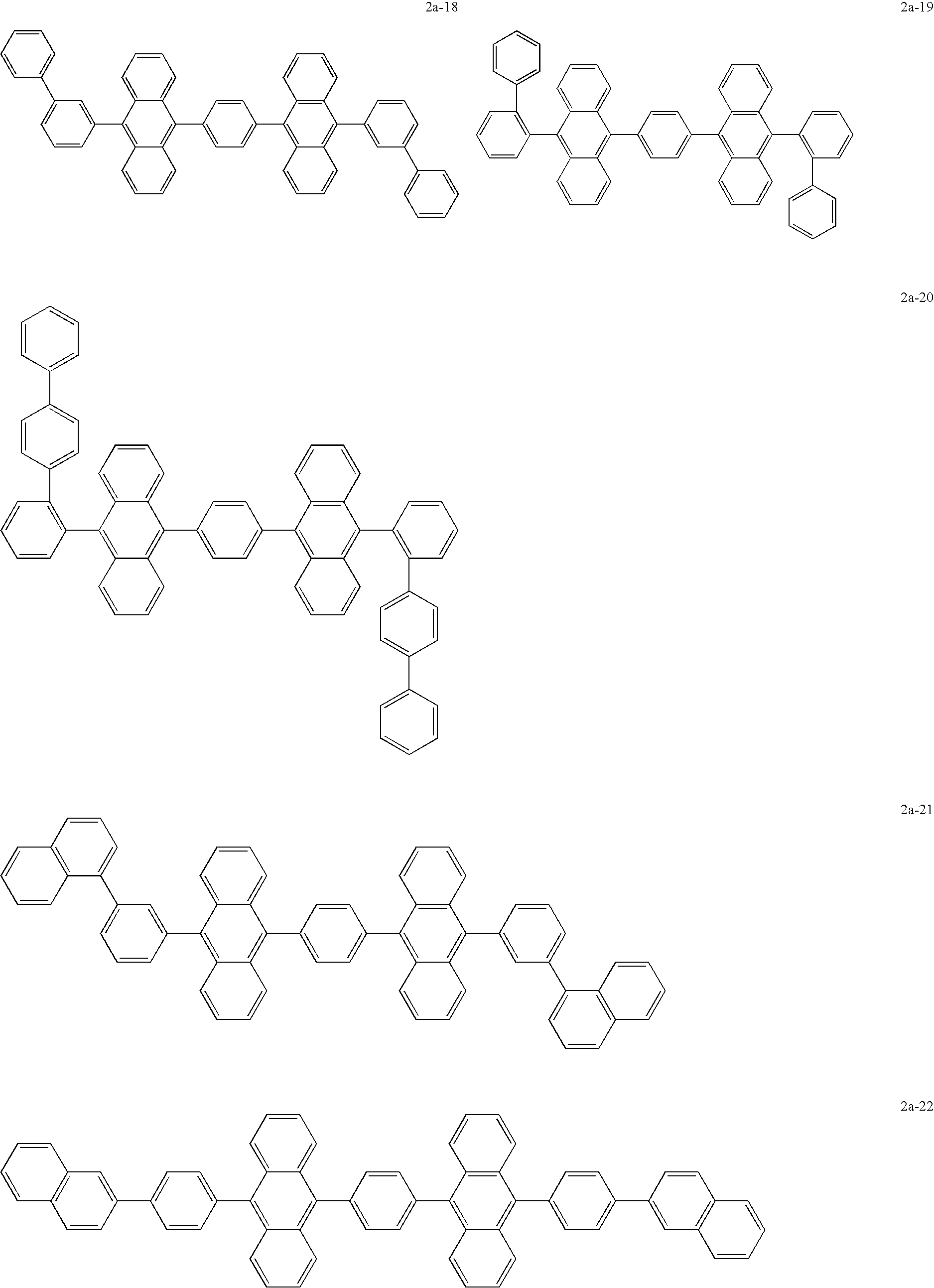

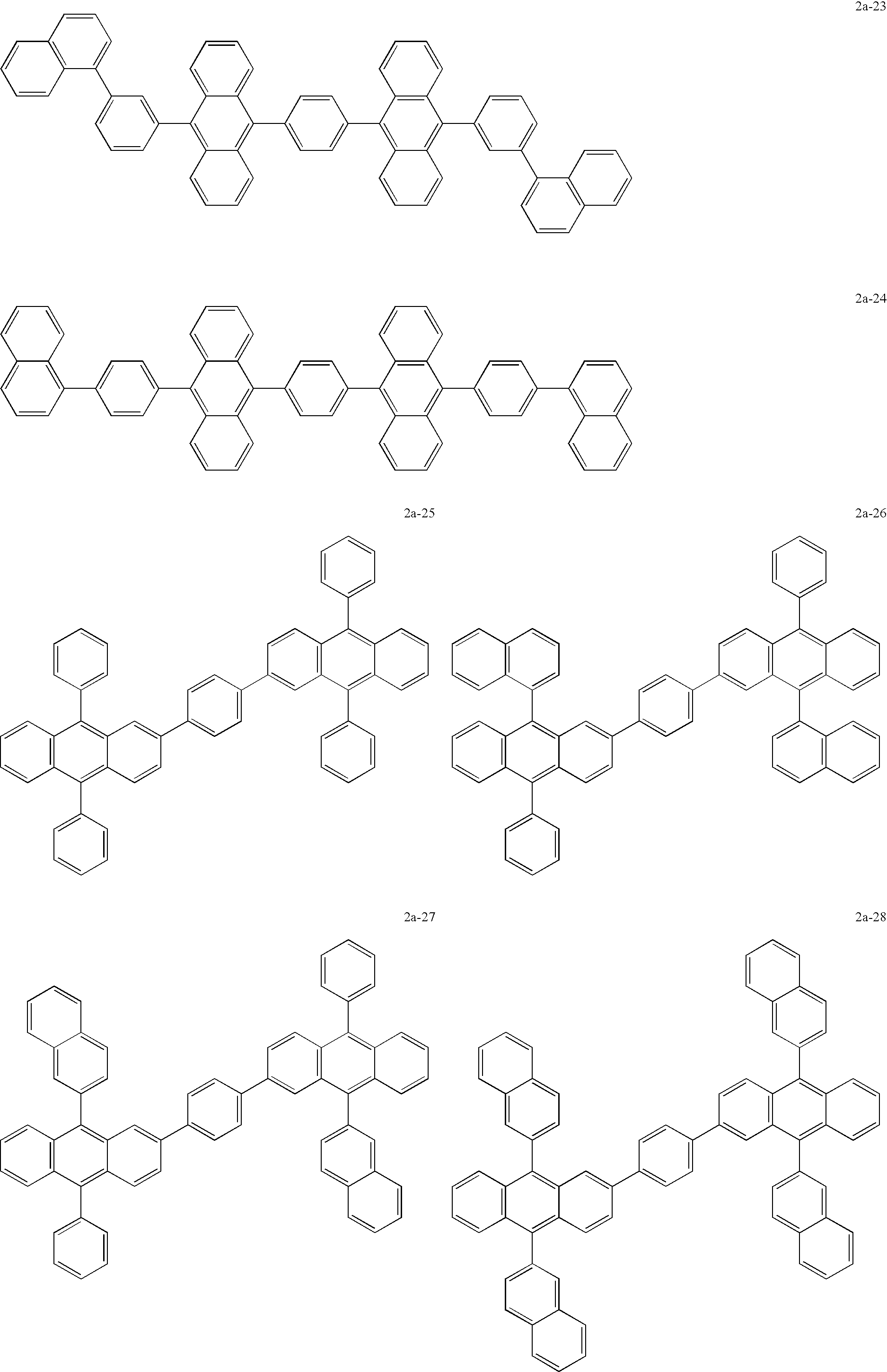

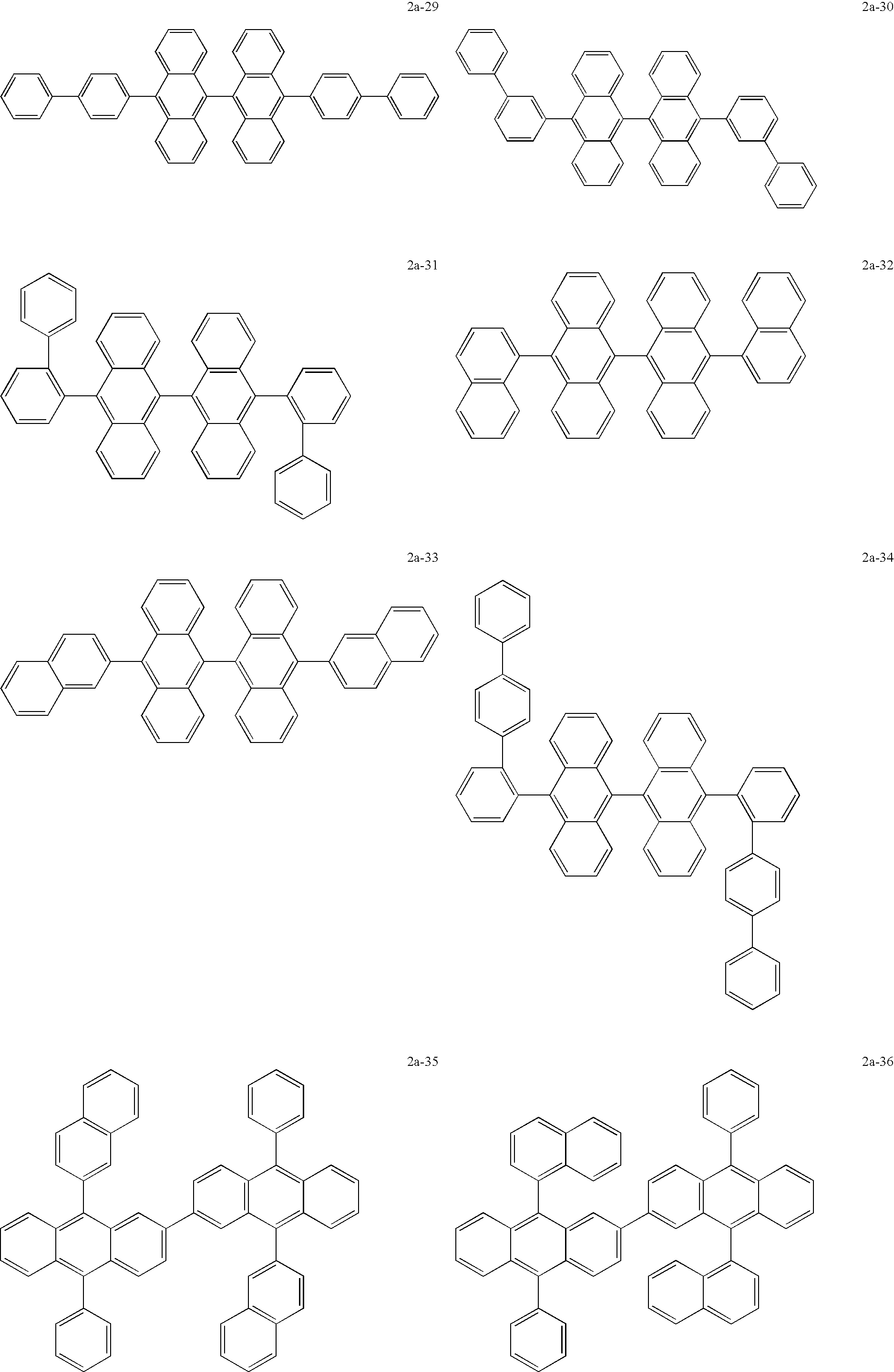

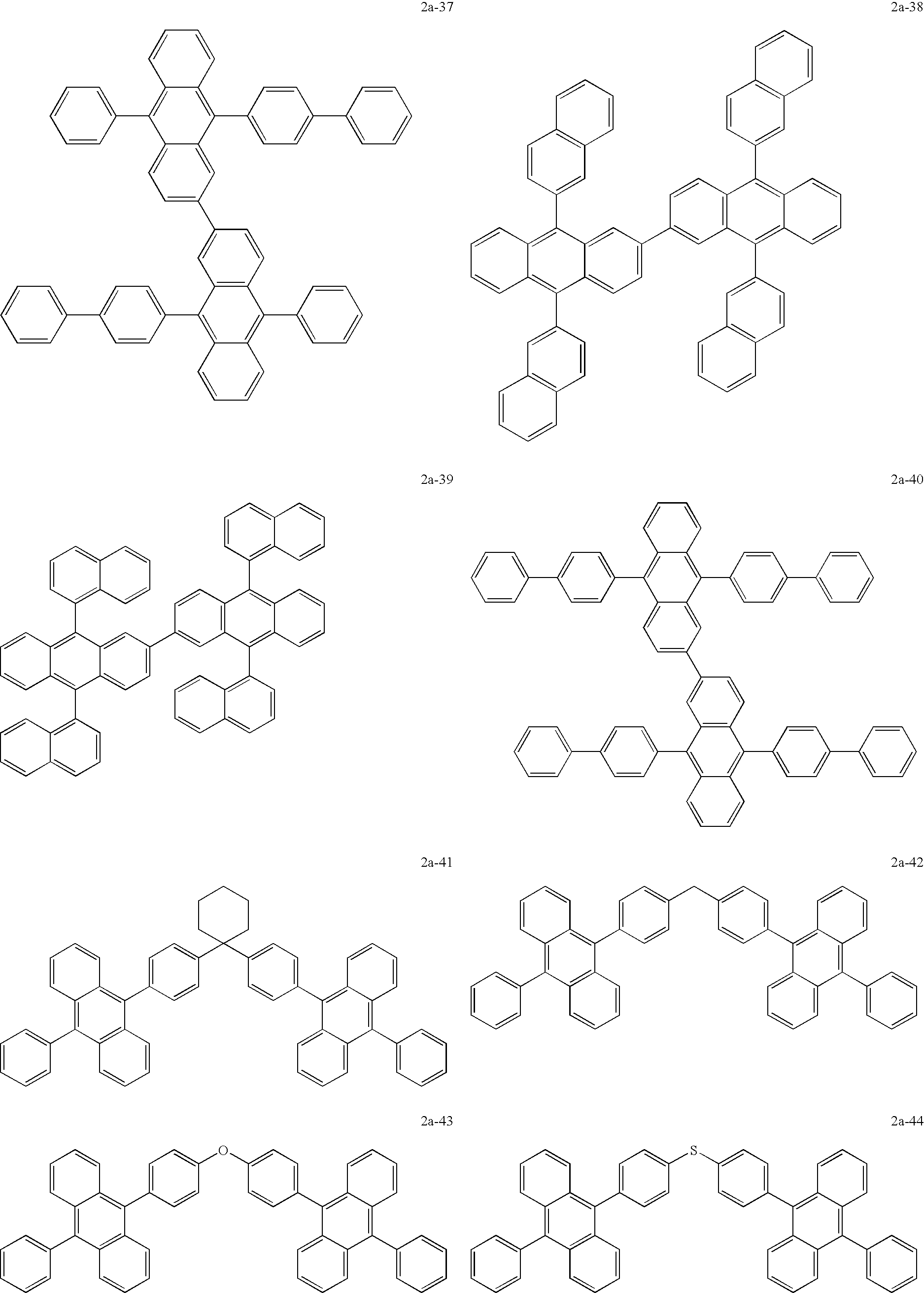

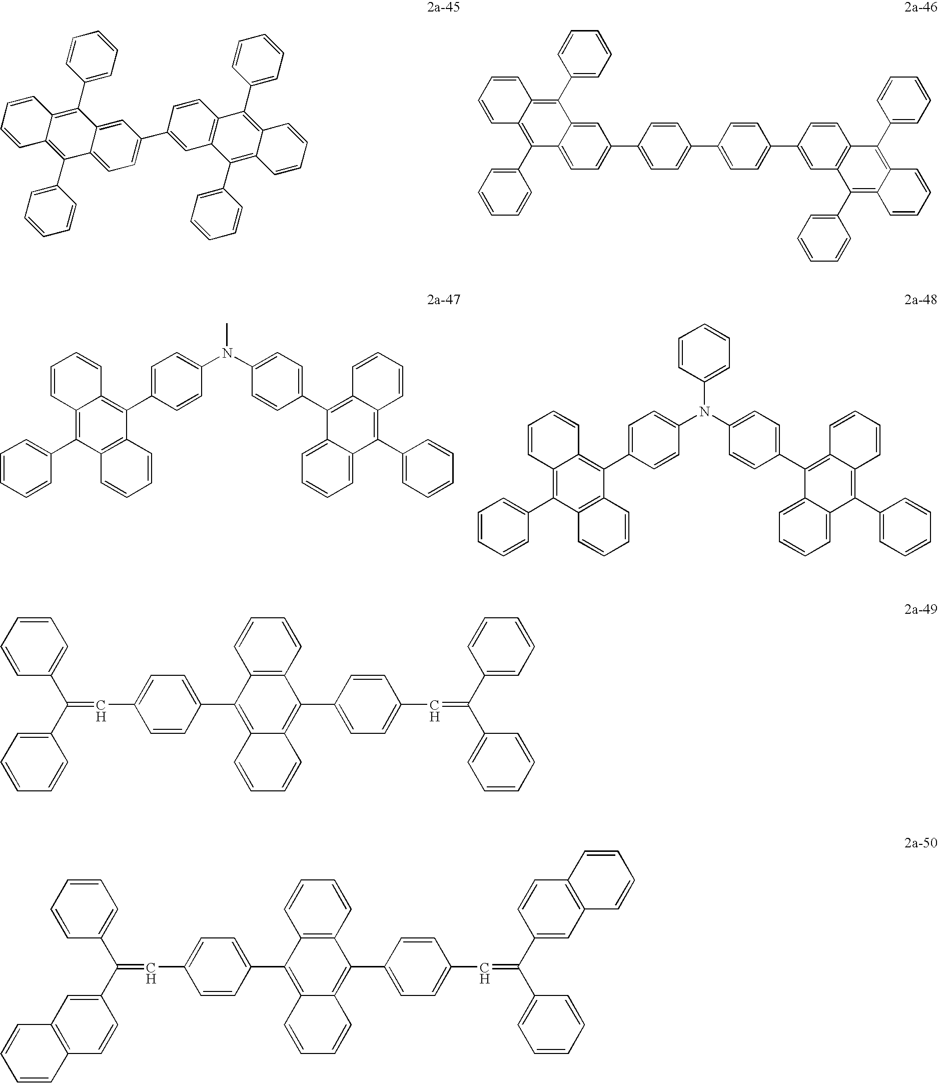

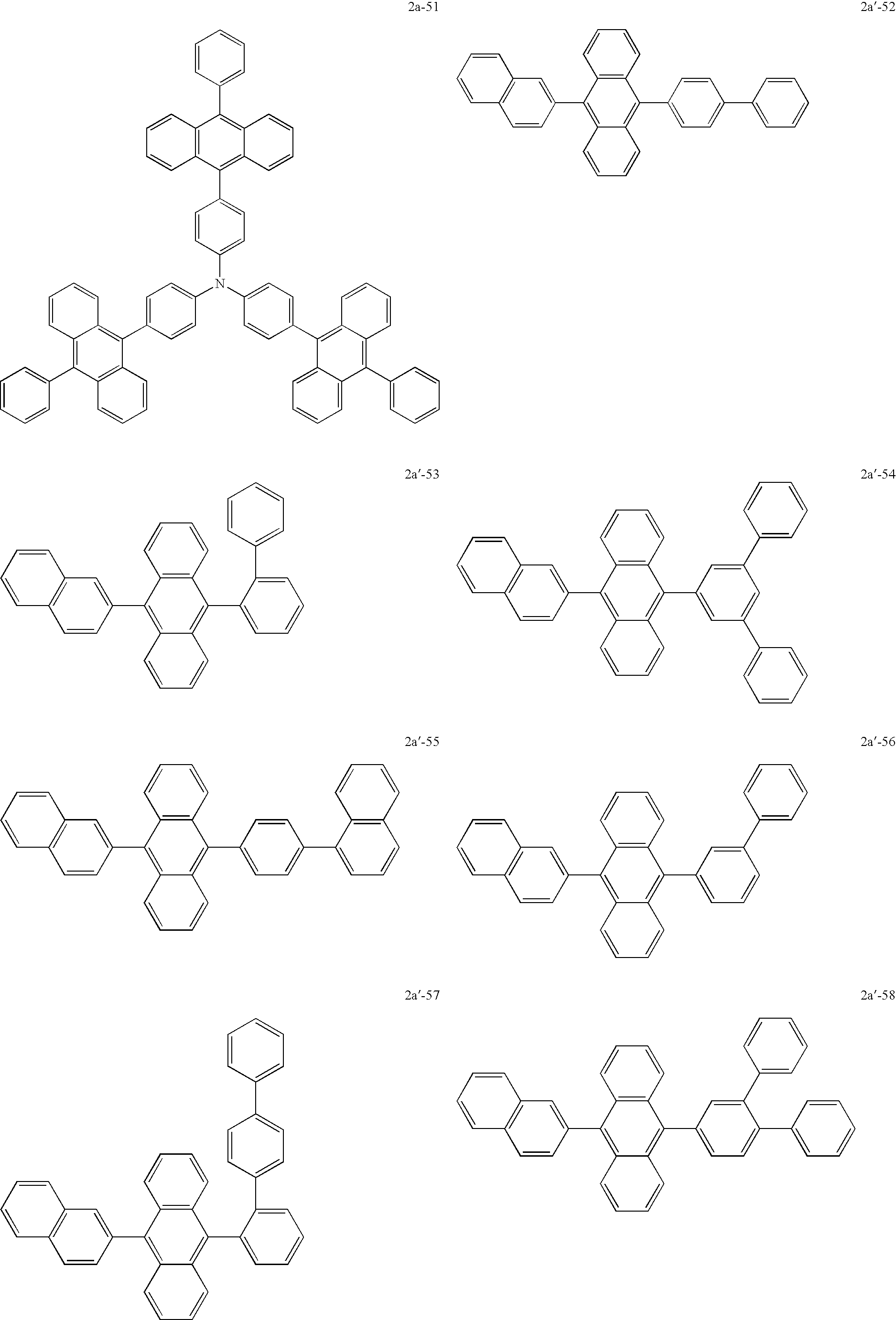

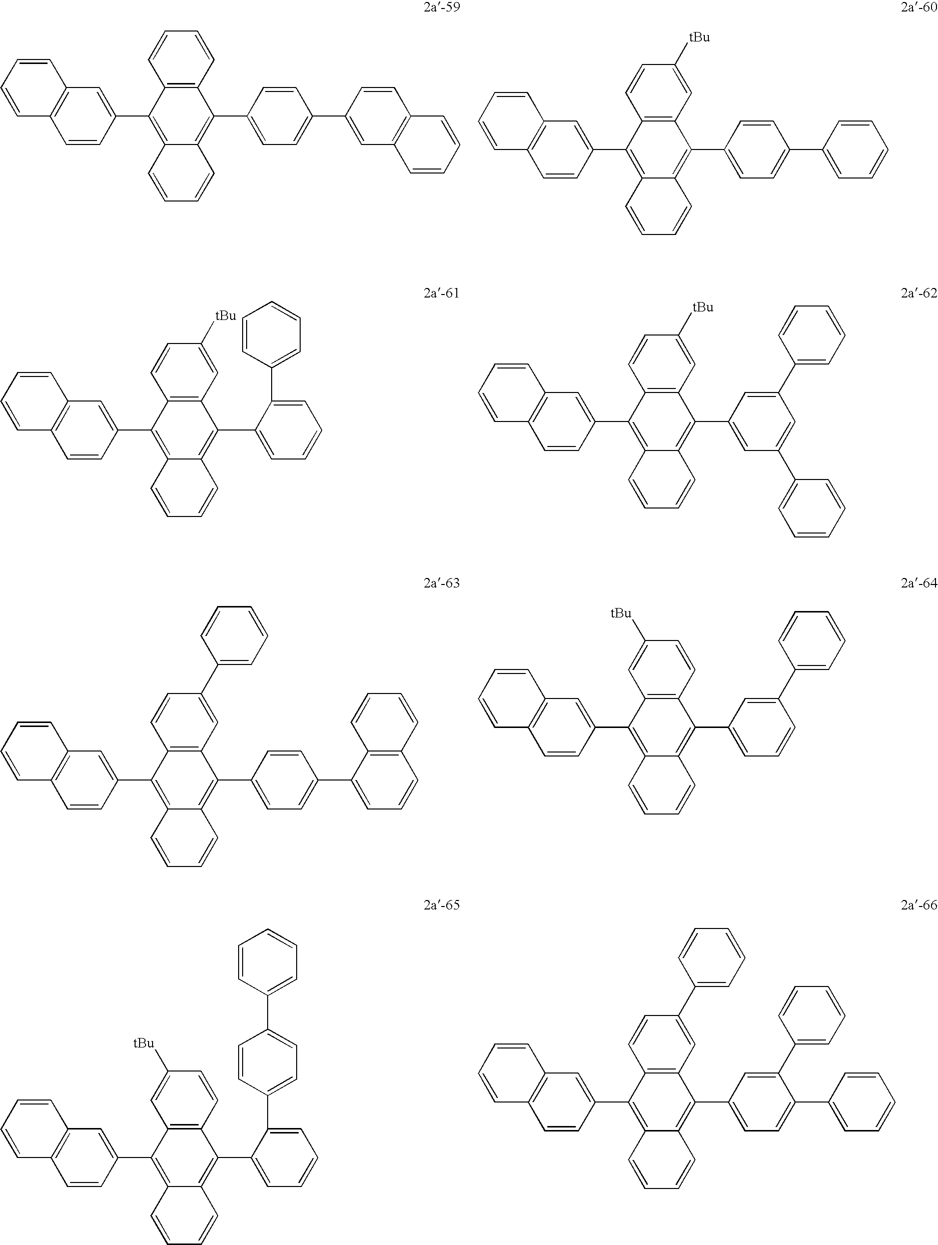

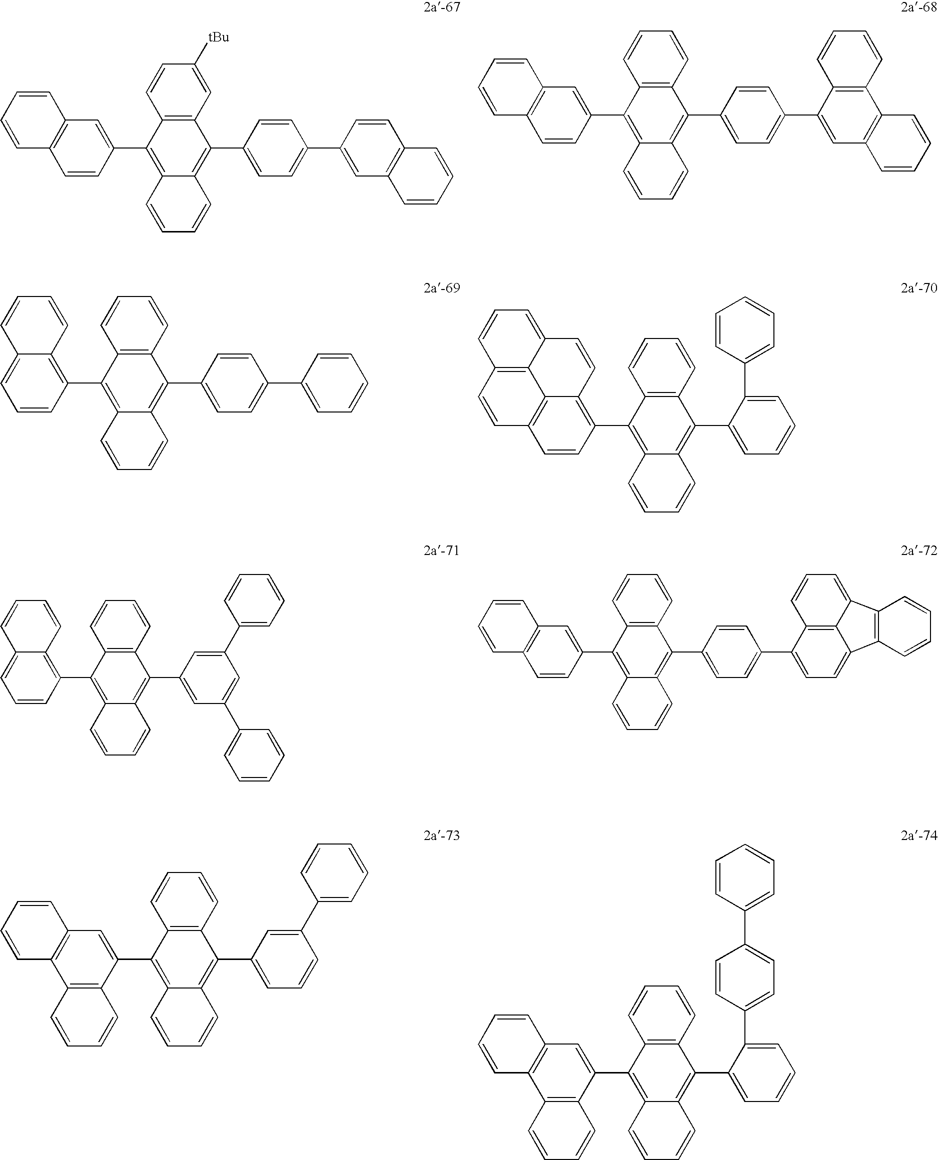

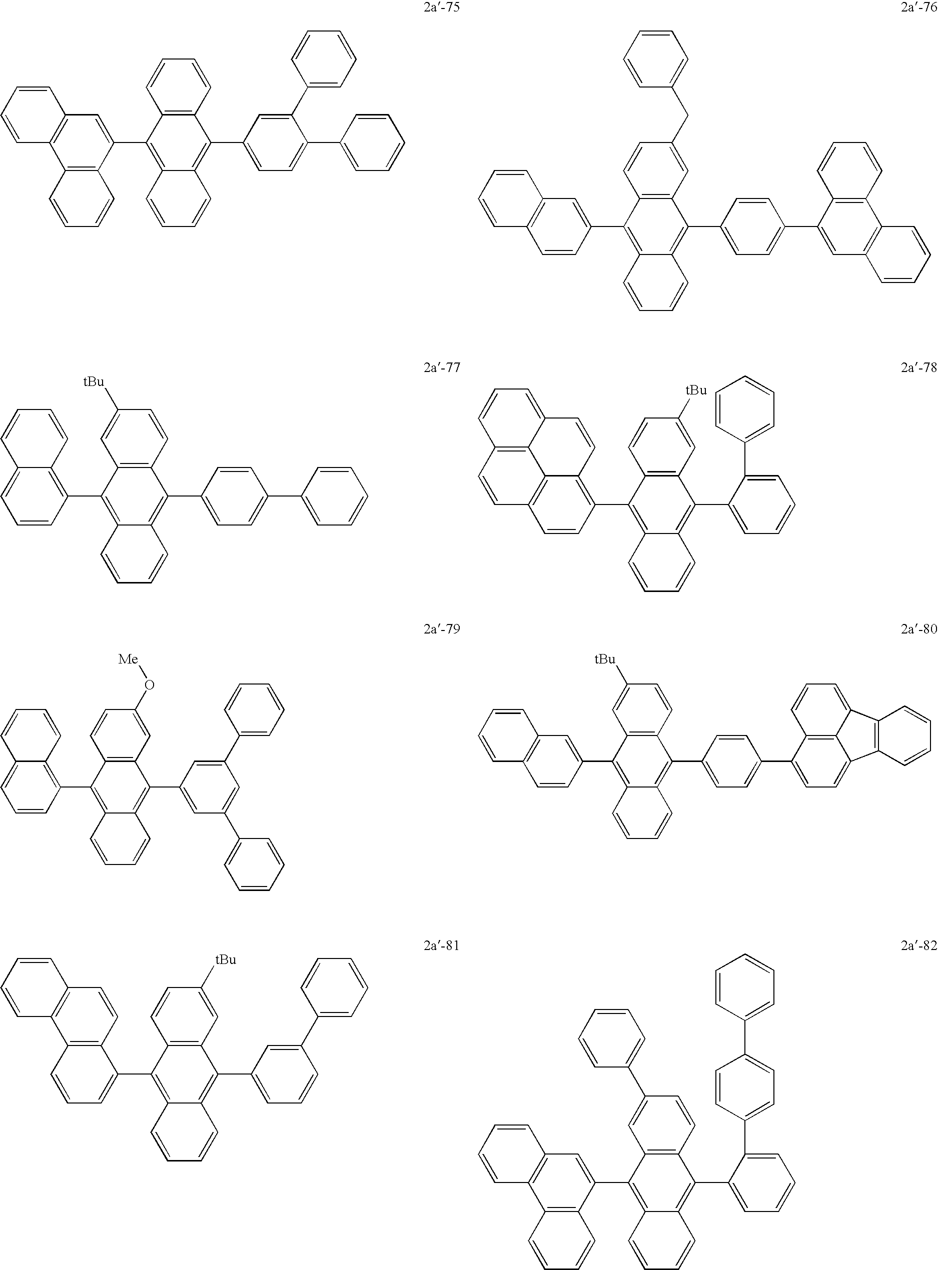

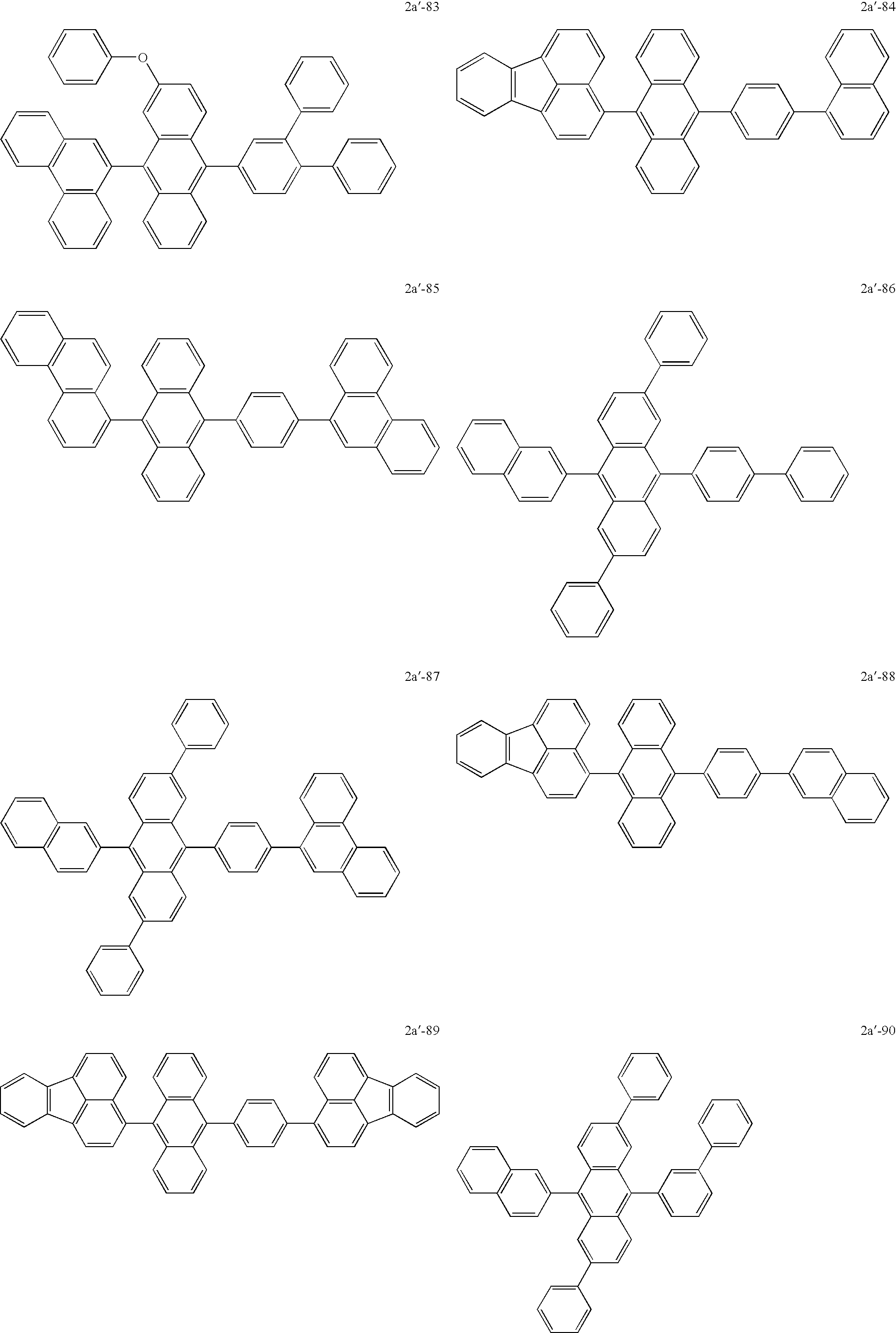

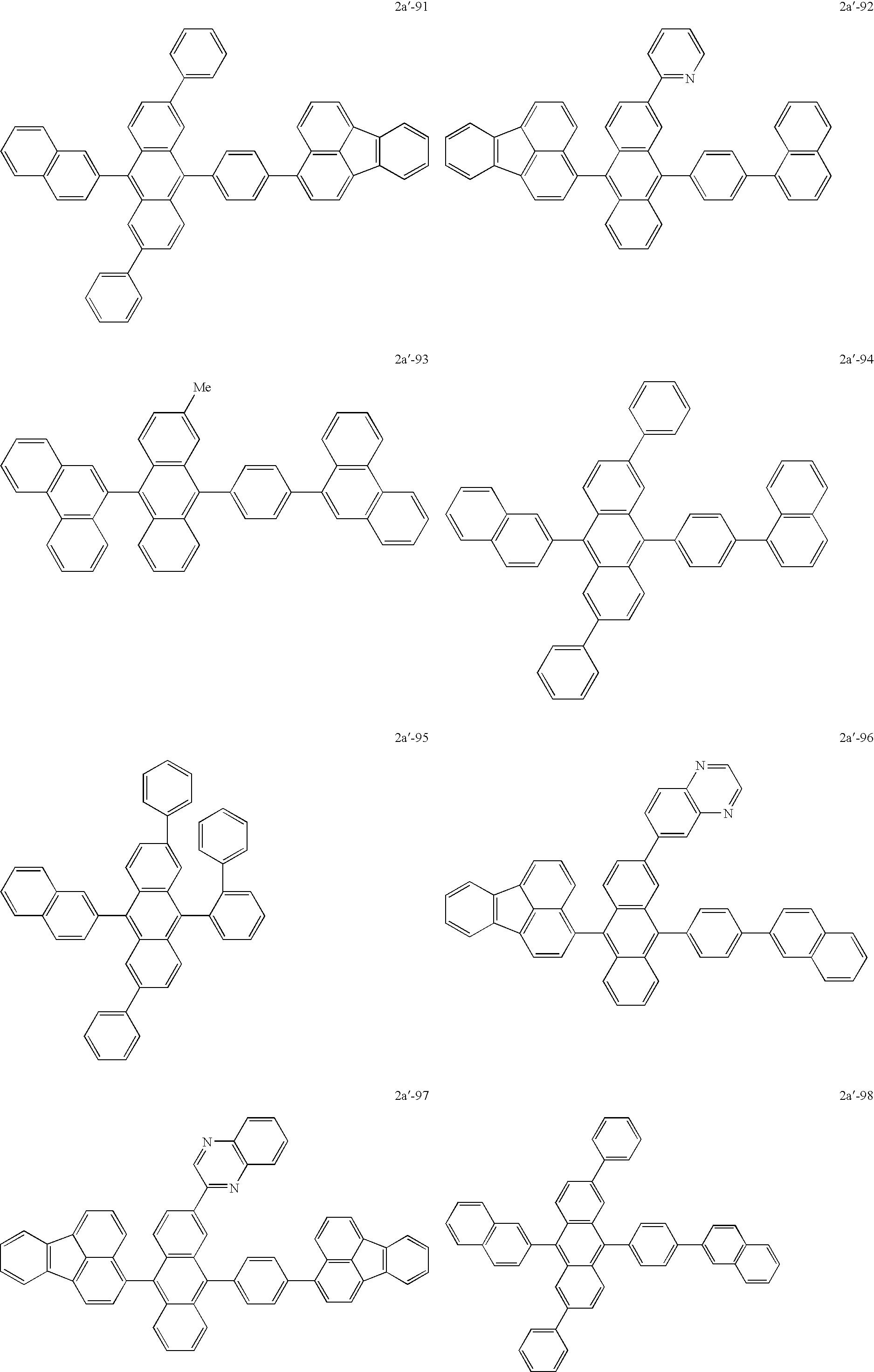

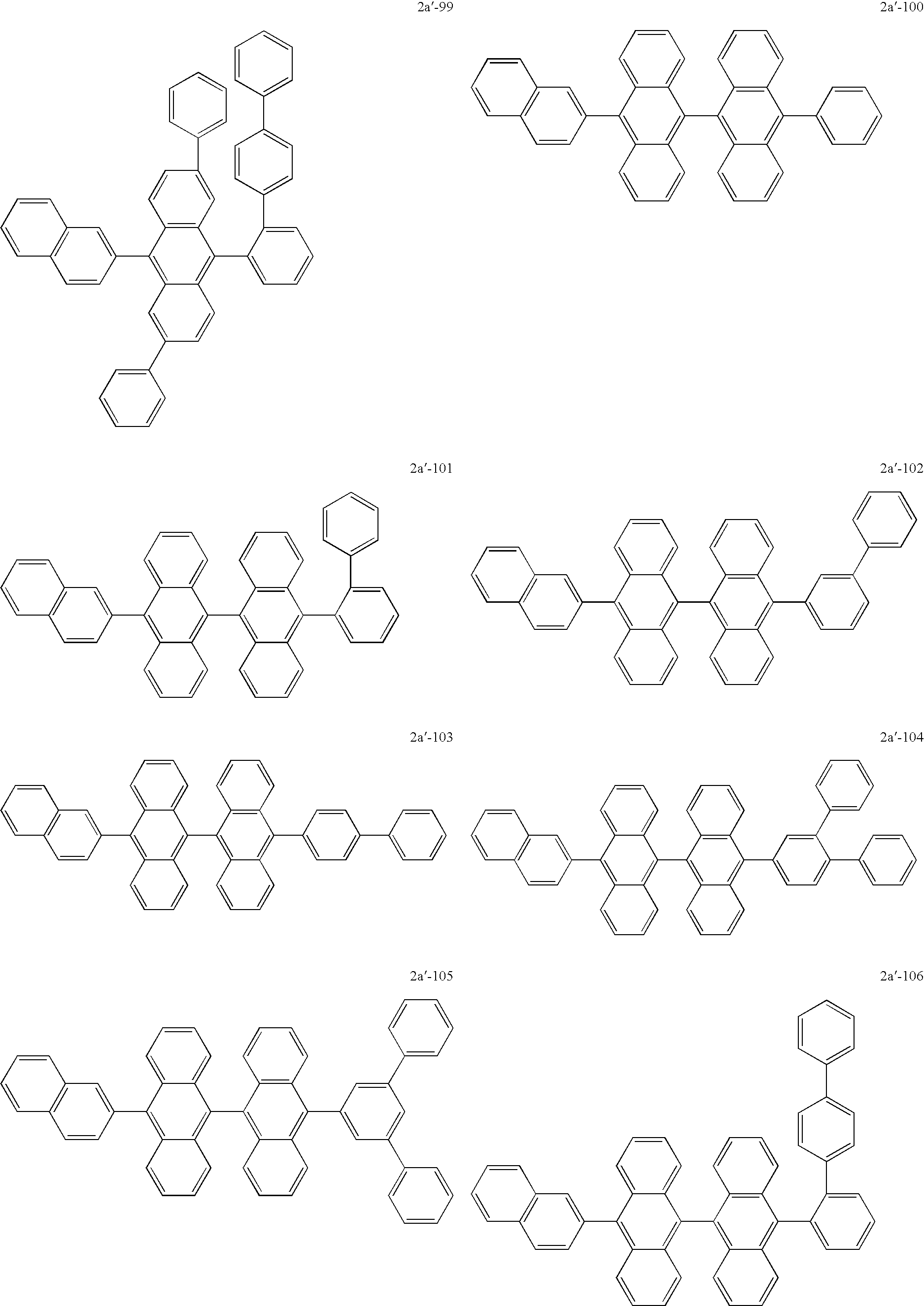

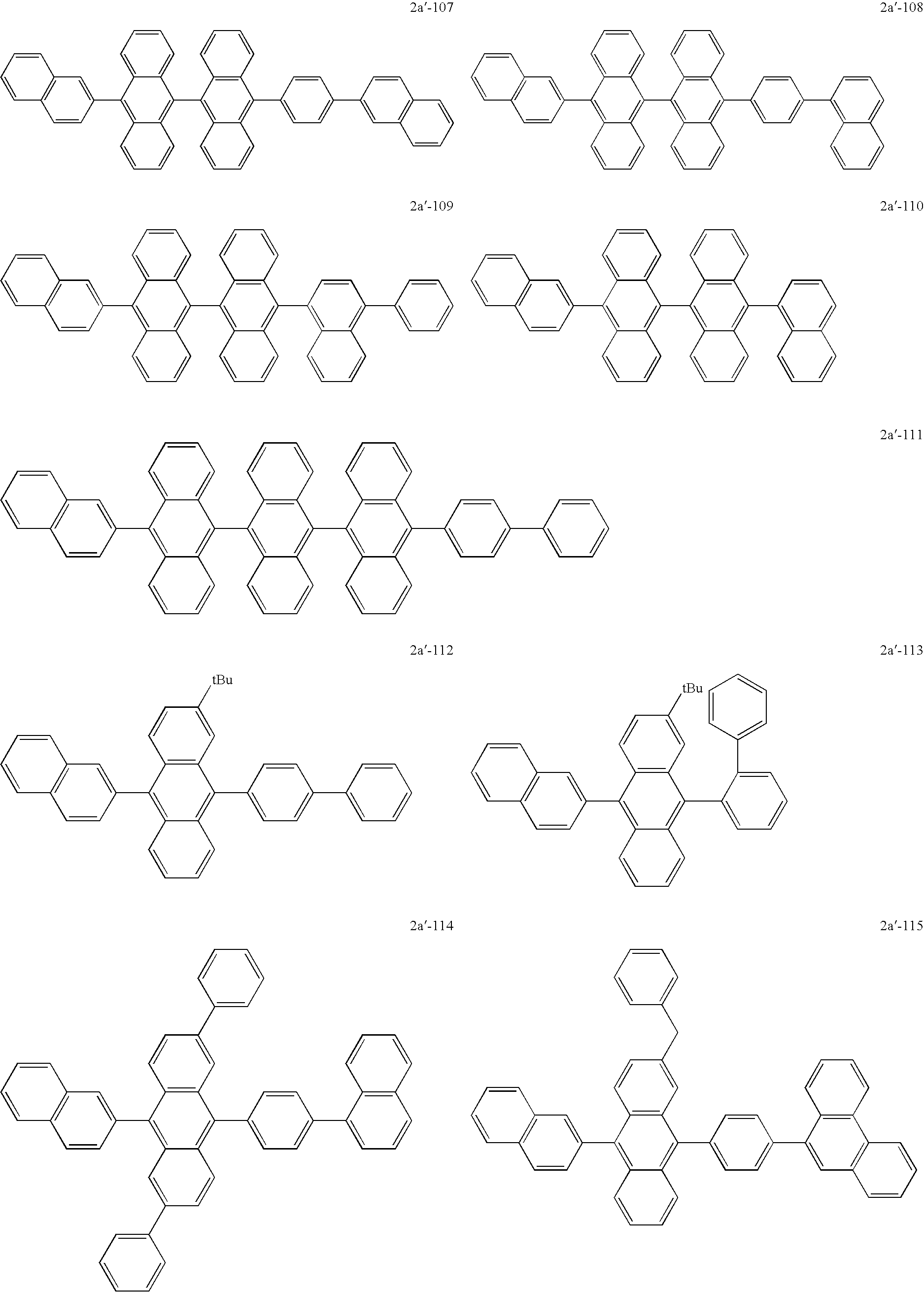

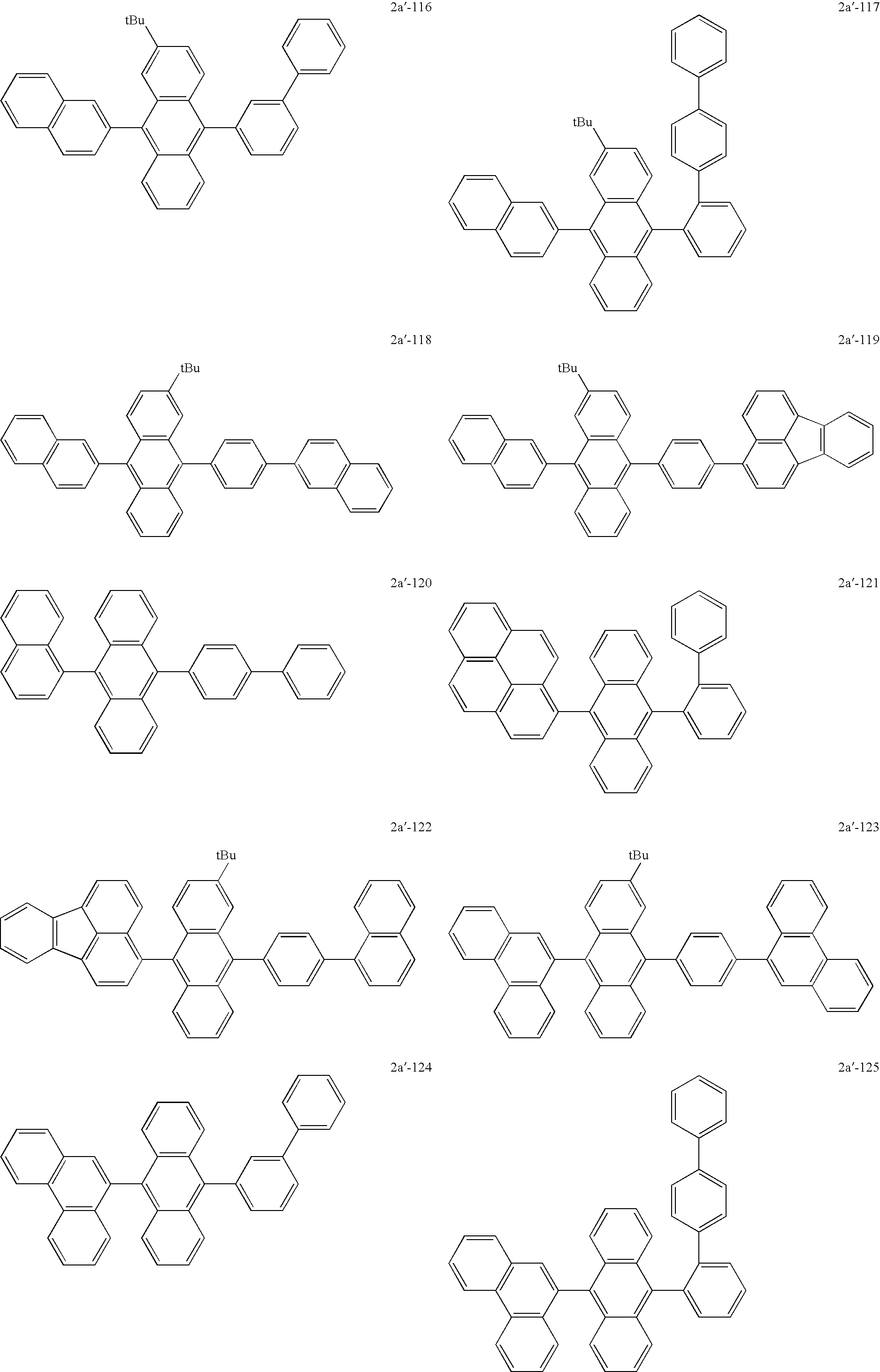

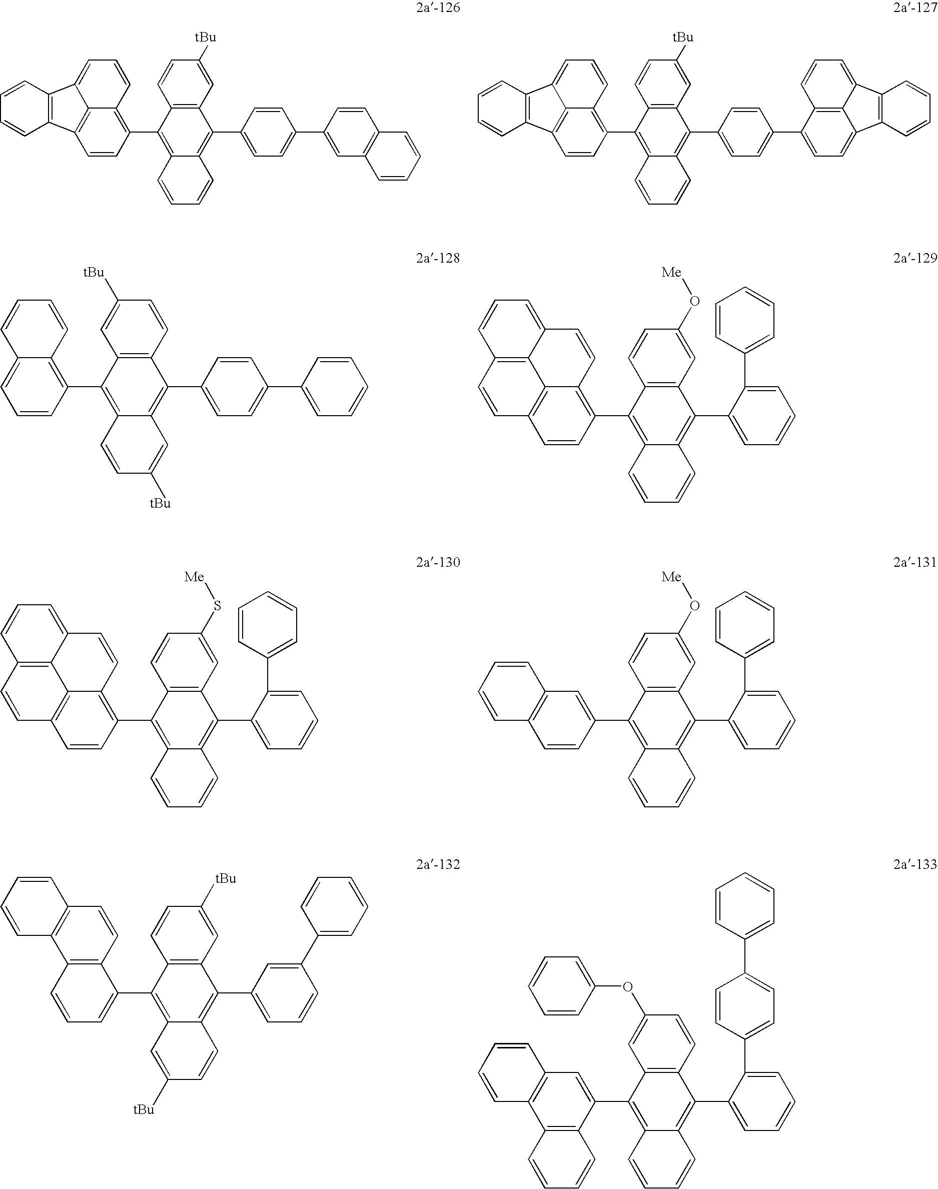

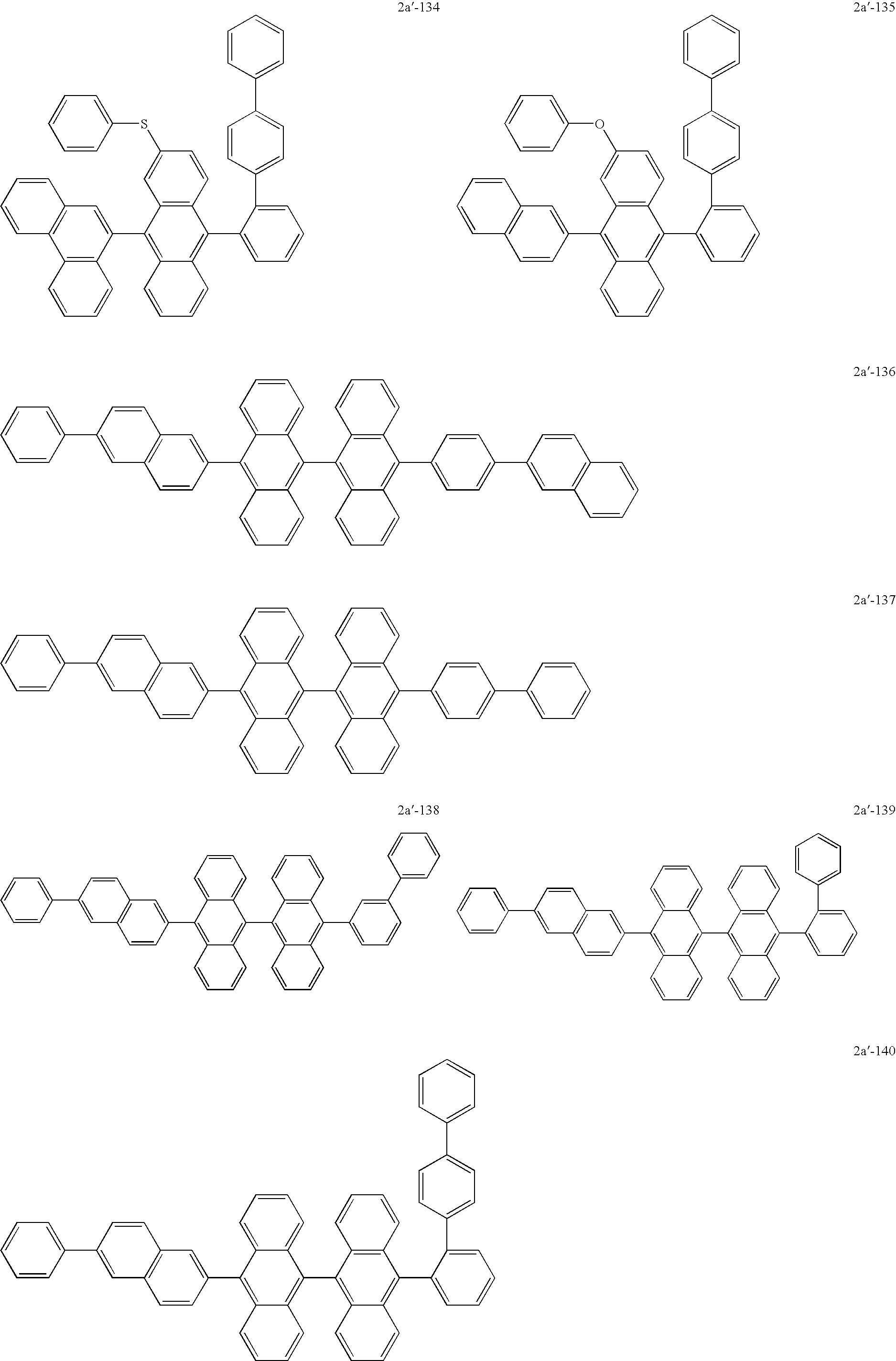

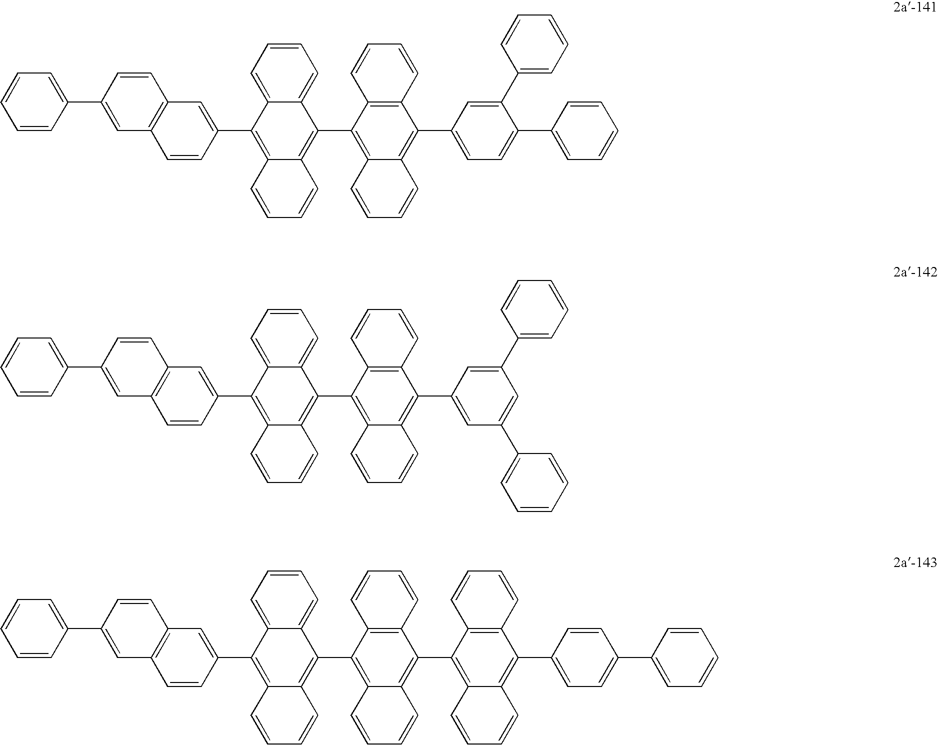

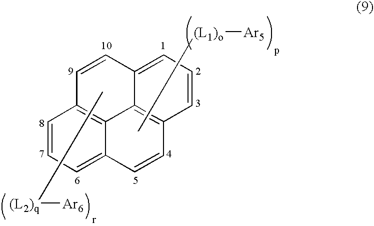

In general formula (9), Ar₅ and Ar₆ each independently represent a substituted or unsubstituted aryl group having 5 to 50 nuclear carbon atoms, and L₁ and L₂ each represent a substituted or unsubstituted phenylene group, a substituted or unsubstituted naphthalenylene group, a substituted or unsubstituted fluorenylene group or a substituted or unsubstituted dibenzosilolylene group;

o represents an integer of 0 to 2, p represents an integer of 1 to 4, q represents an integer of 0 to 2, and r represents an integer of 1 to 4;

the group represented by L₁ or Ar₅ is bonded at one of 1 to 5 positions of pyrene, and the group represented by L₂ or Ar₆ is bonded at one of 6 to 10 positions of pyrene; and

when p+r is an even number, the groups represented by Ar₅, Ar₆, L₁ and L₂ satisfy the following condition (1) or (2):

(1) Ar₅ and Ar₆ represent groups different from each other and/or L₁ and L₂ represent groups different from each other;

(2) when Ar₅ and Ar₆ represent a same group and L₁ and L₂ represent a same group,
(2-1) o and q represent integers different from each other and/or p and r represent integers different from each other, or
(2-2) when o and q represent a same integer and p and r represent a same integer,
a case where positions of substitution of the groups represented by L₁ and L₂ or Ar₅ and Ar₆ on pyrene are 1-position and 6-position, respectively, or 2-position and 7-position, respectively, is excluded when
(2-2-1) the groups represented by L₁ and L₂ or two positions on pyrene are bonded at different bonding positions on the groups represented by Ar₅ and Ar₆, respectively, or
(2-2-2) the groups represented by L₁ and L₂ or two positions on pyrene are bonded at same bonding position on the groups represented by Ar₅ and Ar₆, respectively.

Examples of the pyrene derivative represented by general formula (9) is shown in the following. However, the anthracene derivative is not limited to the compounds shown as the examples.

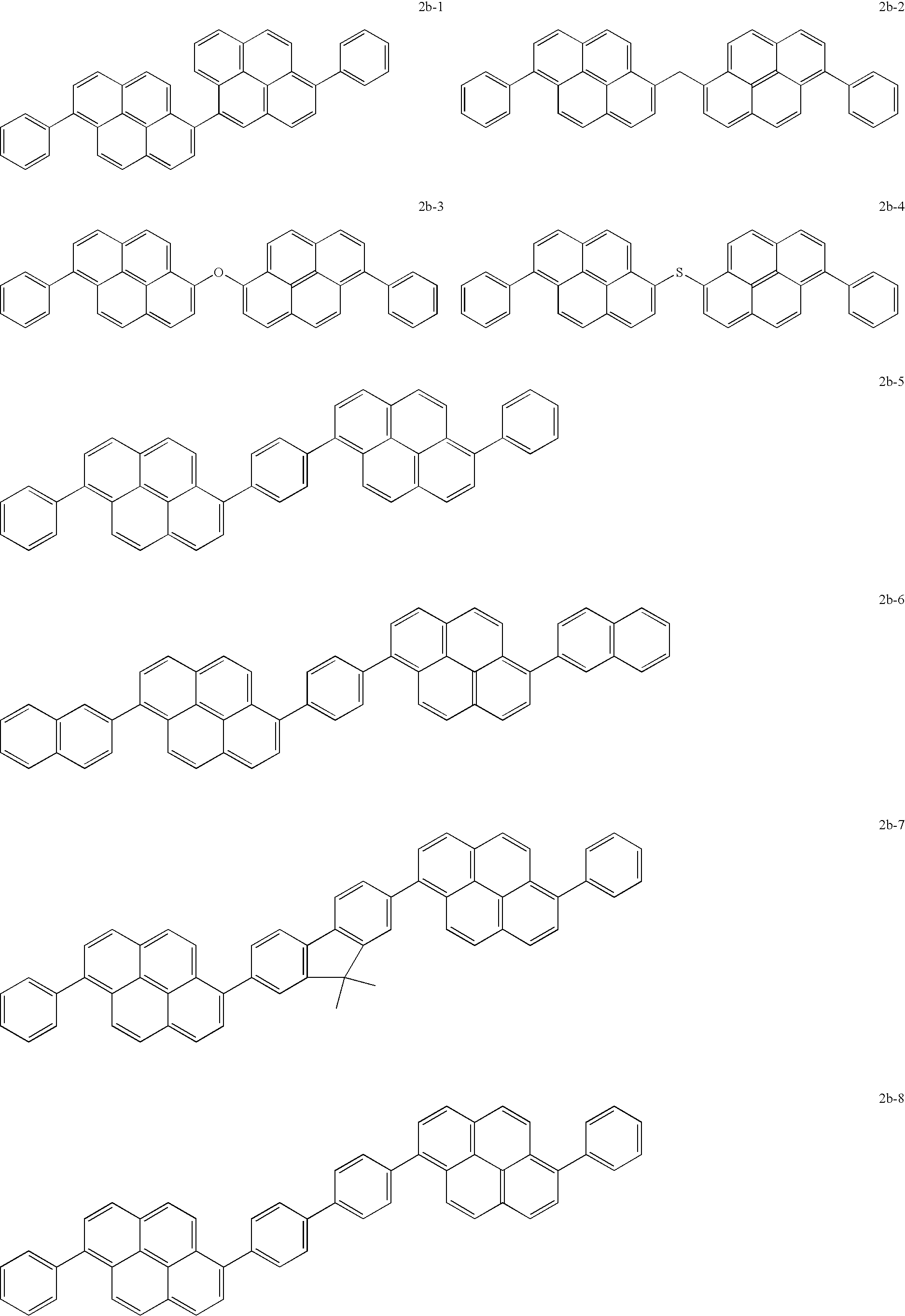

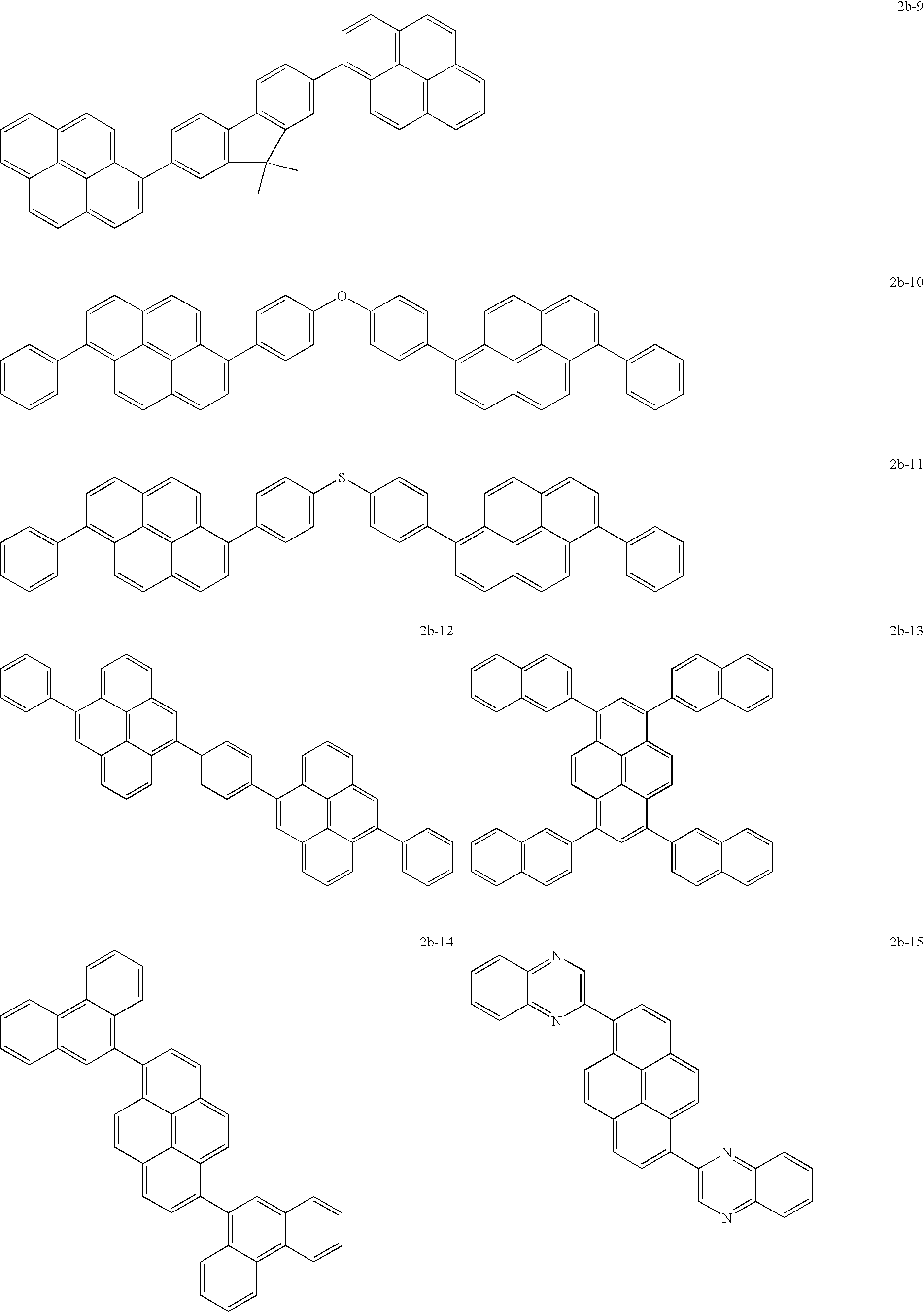

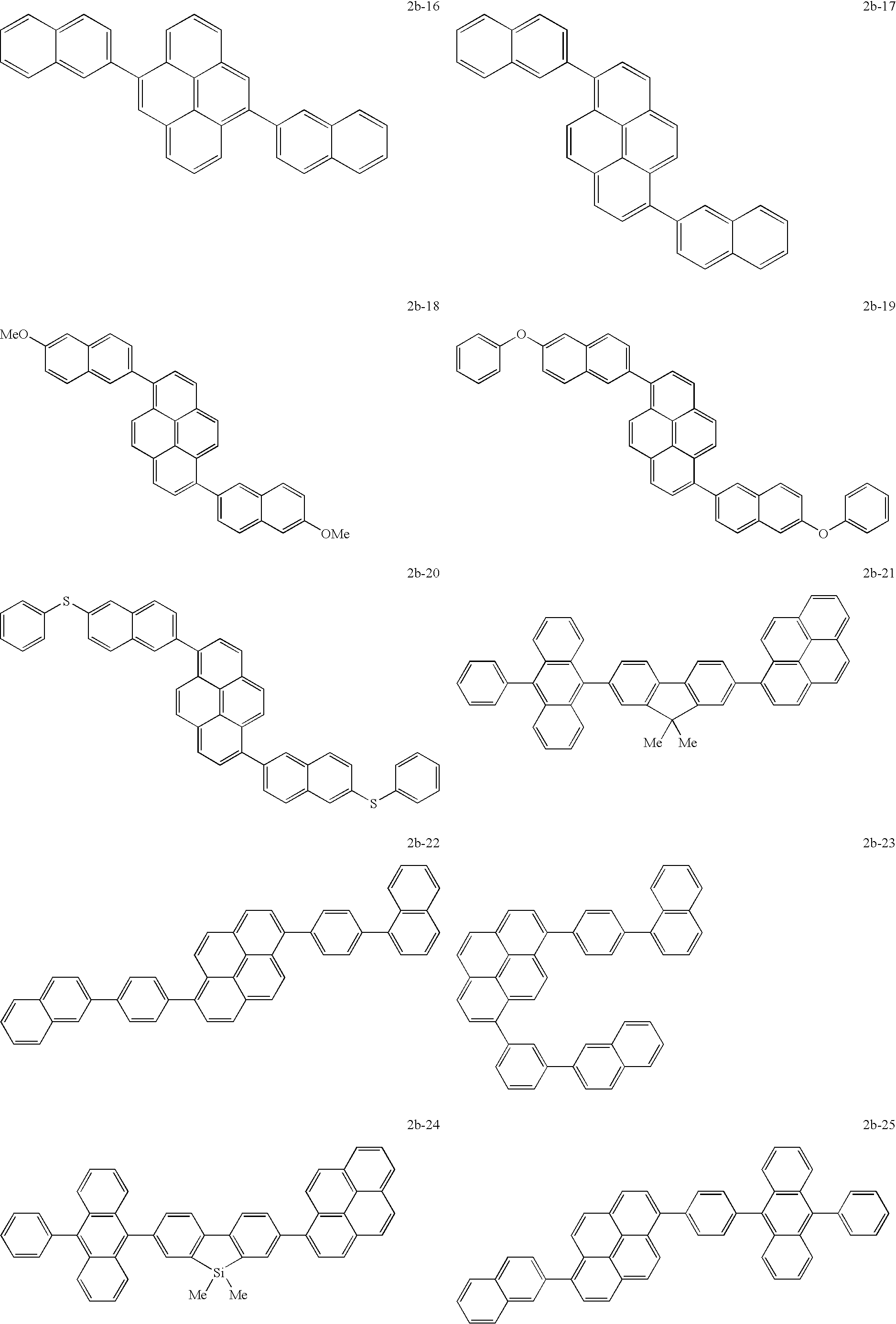

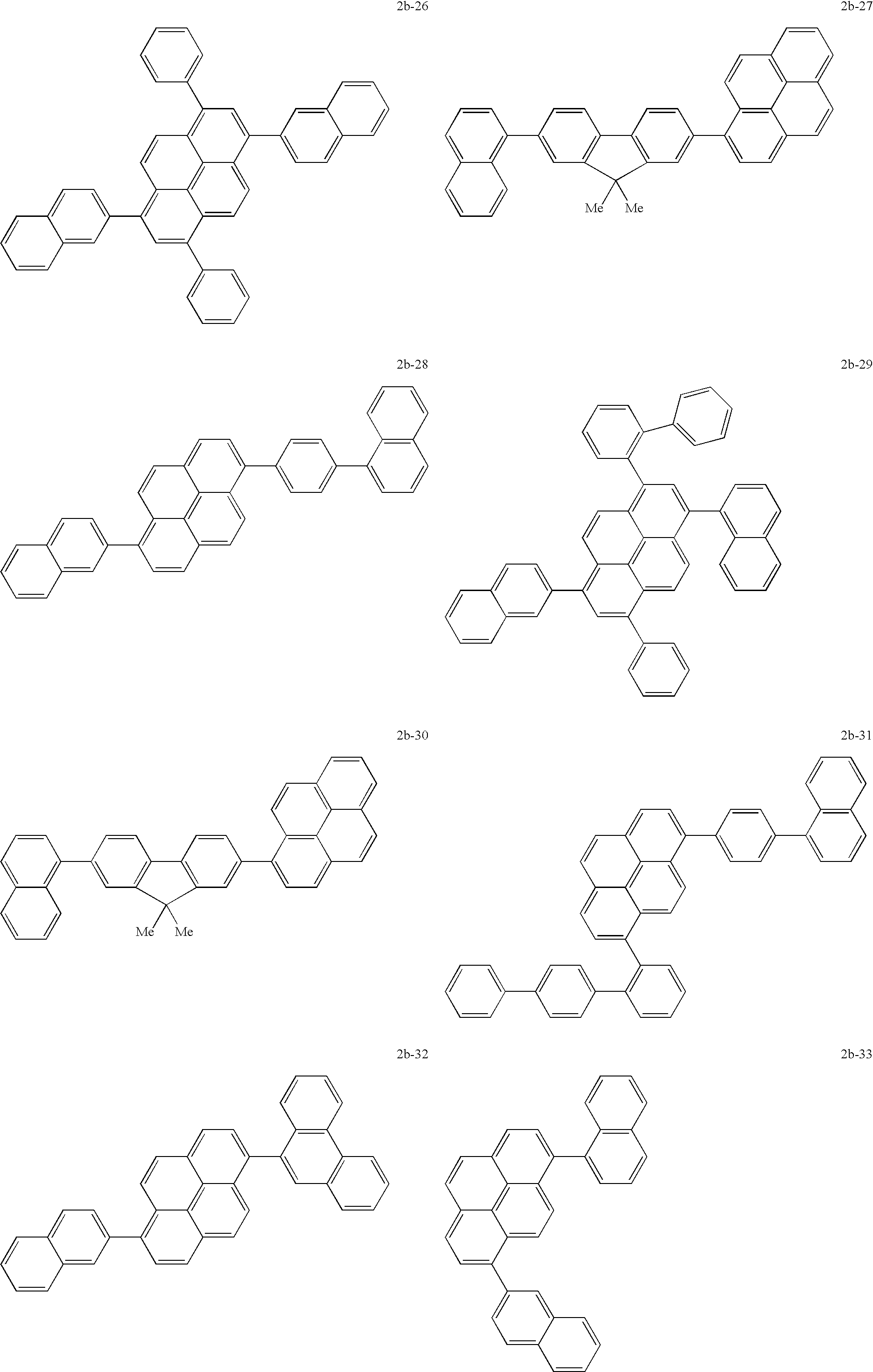

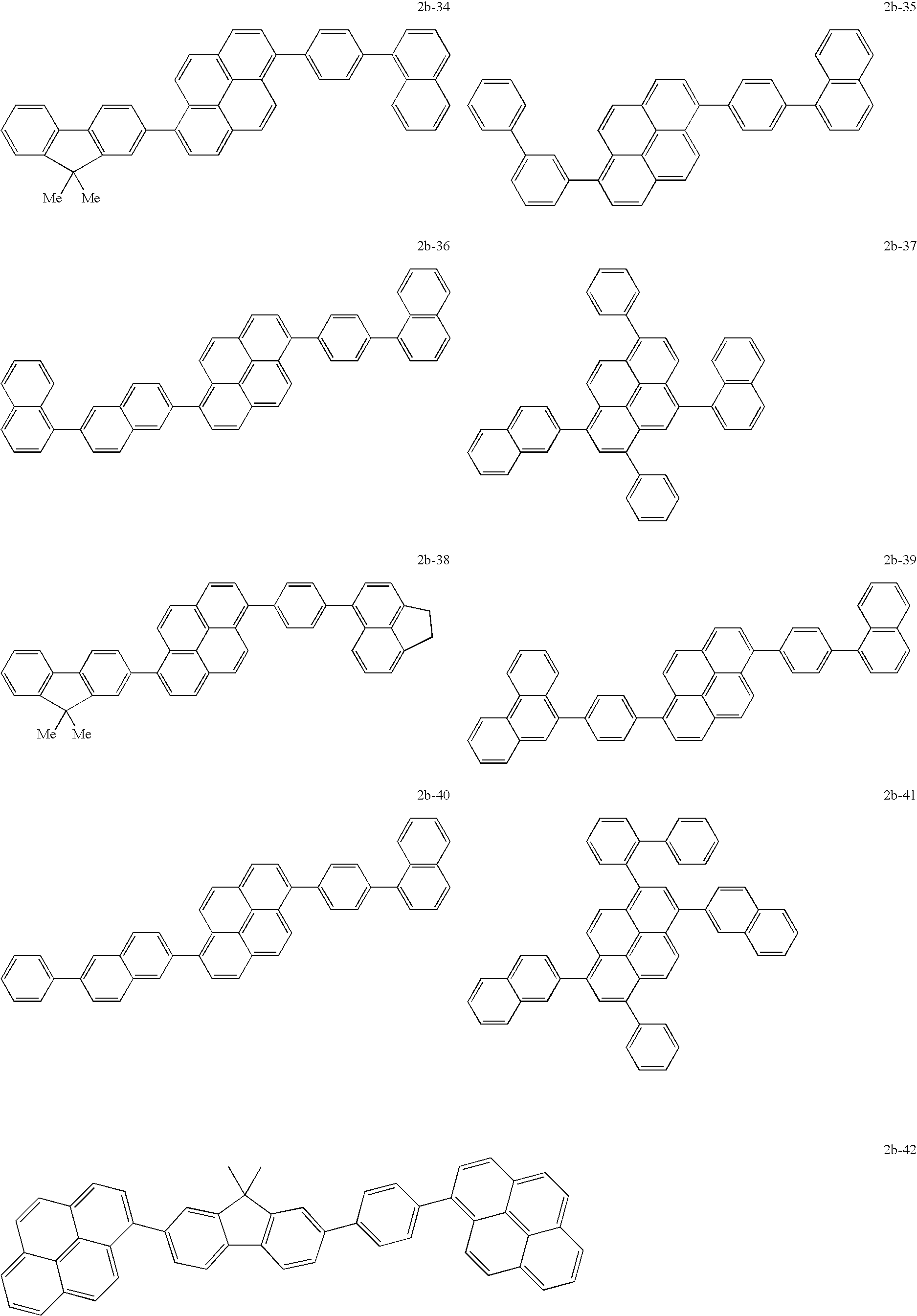

Examples of the groups in the aromatic amine derivatives represented by general formulae (7) to (9) include the groups shown as the examples of the corresponding groups in the compounds represented by general formulae (1) to (4).

In the present invention, examples of the organic EL device in which the organic thin film layer has a plurality of layers include organic EL devices having multi-layer laminate structures of (an anode/a hole injecting layer/a light emitting layer/a cathode), (an anode/a light emitting layer/an electron injecting layer/a cathode) and (an anode/a hole injecting layer/a light emitting layer/an electron injecting layer/a cathode).

Where necessary, conventional light emitting materials, doping materials, hole injecting materials and electron injecting materials may be used in the plurality of layers described above in combination with the aromatic amine derivative of the present invention. Decreases in the luminance and the life of the organic EL device due to quenching can be prevented by using a multi-layer structure for the organic thin film layer. Where necessary, the light emitting materials, the doping materials, the hole injecting materials and the electron injecting materials may be used in combination. By the use of the doping material, the luminance of the emitted light and the efficiency of the light emission can be increased, and red light or blue light can be emitted. The hole injecting layer, the light emitting layer and the electron injecting layer may each have a structure having two or more layers. In this case, in the hole injecting layer, the layer into which holes are injected from the electrode is called the hole injecting layer, and the layer which receives the holes from the hole injecting layer and transports the holes to the light emitting layer is called the hole transporting layer. Similarly, in the electron injecting layer, the layer into which electrons are injected from the electrode is called the electron injecting layer, and the layer which receives the electrons from the electron injecting layer and transports the electrons to the light emitting layer is called the electron transporting layer. These layers are selected and used in accordance with various factors such as the energy level of the material, the heat resistance of the material and the adhesion of the material with the organic layer or the metal electrode.

Examples of the host material and the doping material which are other than the compounds represented by the general formulae (6) to (8) described above and can be used for the light emitting layer in combination with the aromatic amine derivative of the present invention include condensed polycyclic aromatic compounds such as naphthalene, phenanthrene, rubrene, anthracene, tetracene, pyrene, perylene, chrysene, decacyclene, coronene, tetraphenylcyclopentadiene, pentaphenylcyclopentadiene, fluorene, spirofluorene, 9,10-diphenylanthracene, 9,10-bis(phenylethynyl)anthracene, 1,4-bis(9′-ethynylanthracenyl)-benzene; organometallic complex compounds such as tris(8-quinolinolato)aluminum and bis(2-methyl-8-quinolinolato)-4-(phenylphenolinolato)aluminum; triarylamine derivatives, styrylamine derivatives, stilbene derivatives, coumarine derivatives, pyrane derivatives, oxazone derivatives, benzothiazole derivatives, benzoxazole derivatives, benzimidazole derivatives, pyrazine derivatives, cinnamic acid ester derivatives, diketopyrrolopyrrol derivatives, acridone derivatives and quinacridone derivatives. However, the host material and the doping material are not limited to the compounds described above.

As the hole injecting material, compounds which have the ability of transporting holes, exhibit the excellent effect of injection of holes from the anode and the excellent effect of injection of holes to the light emitting layer or the light emitting material, prevent transfer of excimers formed in the light emitting layer to the electron injecting layer or the electron injecting material and have excellent ability of forming a thin film are preferable. Examples of the hole injecting material include phthalocyanine derivatives, naphthalocyanine derivatives, porphyrin derivatives, oxazoles, oxadiazoles, triazoles, imidazoles, imidazolones, imidazolethiones, pyrazolines, pyrazolones, tetrahydroimidazoles, oxazoles, oxadiazoles, hydrazones, acylhydrazones, polyarylalkanes, stilbenes, butadienes, triphenylamines of the benzidine type, triphenylamines of the styrylamine type, triphenylamines of the diamine type, derivatives of these compounds and macromolecular materials such as polyvinylcarbazole, polysilanes and electrically conductive macromolecules. However, the hole injecting material is not limited to the compounds described above.

Among the hole injecting materials which can be used in the organic EL device of the present invention, aromatic tertiary amine derivatives and phthalocyanine derivatives are more effective hole injecting materials.

Examples of the aromatic tertiary amine derivative include triphenylamine, tritolylamine, tolyldiphenylamine, N,N′-diphenyl-N,N′-(3-methylphenyl)-1,1′-biphenyl-4,4′-diamine, N,N,N′,N′-(4-methylphenyl)-1,1′-phenyl-4,4′-diamine, N,N,N′,N′-(4-methylphenyl)-1,1′-biphenyl-4,4′-diamine, N,N′-diphenyl-N,N′-dinaphthyl-1,1′-biphenyl-4,4′-diamine, N,N′-(methylphenyl)-N,N′-(4-n-butylphenyl)phenanthrene-9,10-diamine, N,N-bis(4-di-4-tolylaminophenyl)-4-phenylcyclohexane and oligomer and polymers having the skeleton structure of the above aromatic tertiary amines. However, the aromatic tertiary amine derivative is not limited to the compounds described above.

Examples of the phthalocyanine (Pc) derivative include phthalocyanine derivatives and naphthalocyanine derivatives such as H₂Pc, CuPc, CoPc, NiPc, ZnPc, PdPc, FePc, MnPc, ClAlPc, ClGaPc, ClInPc, ClSnPc, Cl₂SiPc, (HO)AlPc, (HO)GaPc, VOPc, TiOPc, MoOPc, GaPc-O—GaPc. However, the phthalocyanine derivative is not limited to the compounds described above.

In the organic EL device of the present invention, it is preferable that the layer comprising the aromatic amine derivative and/or the phthalocyanine derivative described above such as the hole transporting layer or the hole injecting layer described above is disposed between the light emitting layer and the anode.

As the electron injecting material, compounds which have the ability of transporting electrons, exhibit the effect of injection of electrons from the cathode and the excellent effect of injection of electrons into the light emitting layer or the light emitting material, prevent transfer of excimers formed in the light emitting layer into the hole injecting layer and have excellent ability of forming a thin film, are preferable. Examples of the electron injecting material include fluorenone, anthraquinodimethane, diphenoquinones, thiopyrane dioxides, oxazoles, oxadiazoles, triazoles, imidazoles, perylenetetracarboxylic acid, fluorenylidenemethane, anthraquinodimethane, anthrone and derivatives of these compounds. However, the electron injecting material is not limited to the compounds described above. The sensitivity of the hole injecting material can be improved by adding an electron-accepting substance, and the sensitivity of the electron injecting material can be improved by adding an electron-donating substance.

In the organic EL device of the present invention, more effective electron injecting materials are metal complex compounds and five-membered cyclic derivatives having nitrogen atom.

Examples of the metal complex compound include 8-hydroxyquinolinatolithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato) aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum and bis(2-methyl-8-quinolinato)(2-naphtholato)gallium. However, the metal complex compound is not limited to the compounds described above.

As the five-membered cyclic derivative having nitrogen atom, for example, derivatives of oxazole, thiazole, oxadiazole, thiadiazole and triazole are preferable. Examples of the five-membered cyclic derivative having nitrogen atom include 2,5-bis(1-phenyl)-1,3,4-oxazole, dimethylPOPOP, 2,5-bis(1-phenyl)-1,3,4-thiazole, 2,5-bis(1-phenyl)-1,3,4-oxadiazole, 2-(4′-tert-butylphenyl)-5-(4″-biphenyl)-1,3,4-oxadiazole, 2,5-bis(1-naphthyl)-1,3,4-oxadiazole, 1,4-bis[2-(5-phenyloxadiazolyl)]benzene, 1,4-bis[2-(5-phenyloxadiazolyl)-4-tert-butylbenzene], 2-(4′-tert-butyl-phenyl)-5-(4″-biphenyl)-1,3,4-thiadiazole, 2,5-bis(1-naphthyl)-1,3,4-thiazole, 1,4-bis-[2-(5-phenylthiadiazolyl)]benzene, 2-(4′-t-butylphenyl)-5-(4′-biphenyl)-1,3,4-triazole, 2,5-bis(1-naphthyl)-1,3,4-triazole and 1,4-bis-[2-(5-phenyltriazolyl)]benzene. However, the five-membered cyclic derivative having nitrogen atom is not limited to the compounds described above.

In the organic EL device of the present invention, the light emitting layer may further comprise at least one of light emitting materials, doping materials, hole injecting materials and electron injecting materials in the same layer in addition to at least one of the aromatic amine derivatives represented by general formulae (1) to (5). A protective layer may be formed on the surface of the device, or the entire device may be protected with a silicone oil or a resin so that stability of the organic EL device obtained in accordance with the present invention with respect to the temperature, the moisture and the atmosphere is improved.

As the electrically conductive material used for the anode in the organic EL device of the present invention, a material having a work function greater than 4 eV is suitable. Carbon, aluminum, vanadium, iron, cobalt, nickel, tungsten, silver, gold, platinum, palladium, alloys of these metals, metal oxides such as tin oxide and indium oxide used for ITO substrates and NESA substrates, and organic electrically conductive resins such as polythiophene and polypyrrol can be used. As the electrically conductive material used for the cathode, a material having a work function smaller than 4 eV is suitable. Magnesium, calcium, tin, lead, titanium, yttrium, lithium, ruthenium, manganese, aluminum, lithium fluoride and alloys of these metals can be used, but the material is not limited to these materials. Typical examples of the alloy include magnesium/silver, magnesium/indium and lithium/aluminum, but the alloy is not limited to these alloys. The ratio of the amounts of the components in the alloy is controlled by the temperature of the sources of vapor deposition, the atmosphere and the degree of vacuum and adjusted to a suitable value. The anode and the cathode may have a structure having two or more layers, where necessary.

In the organic EL device of the present invention, it is desirable that at least one face of the device is sufficiently transparent in the wavelength range of the light emitted from the device so that the emitted light is efficiently obtained. It is desirable that the substrate is transparent. The transparent electrode is formed in accordance with a process such as the vapor deposition process or the sputtering process using the above electrically conductive material in a manner such that the prescribed transparency is assured. It is preferable that the electrode on the light emitting face has a transmittance of light of 10% or greater. The substrate is not particularly limited as long as the substrate has sufficient mechanical and thermal strength and transparency. Examples of the substrate include glass substrates and transparent resin films. Examples of the transparent resin films include films of polyethylene, ethylene-vinyl acetate copolymers, ethylene-vinyl alcohol copolymers, polypropylene, polystyrene, polymethyl methacrylate, polyvinyl chloride, polyvinyl alcohol, polyvinyl butyral, nylons, polyether ether ketones, polysulfones, polyether sulfones, tetrafluoroethylene-perfluoroalkyl vinyl ether copolymers, polyvinyl fluoride, tetrafluoroethylene-ethylene copolymers, tetrafluoroethylene-hexafluoropropylene copolymers, polychlorotrifluoroethylene, polyvinylidene fluoride, polyesters, polycarbonates, polyurethanes, polyimides, polyether imides, polyimides and polypropylene.

To the formation of the layers in the organic EL device of the present invention, any of dry film forming processes such as the vacuum vapor deposition process, the sputtering process, the plasma process and the ion plating process and wet film forming processes such as the spin coating process, the dipping process and the flow coating process can be applied. It is necessary that the thickness of the film is set at a suitable value although the thickness of the layer is not particularly limited. When the thickness is excessively great, a great voltage must be applied to obtain the prescribed output, and the efficiency decreases. When the thickness is excessively small, defects such as pin holes are formed, and the sufficient luminance of the emitted light cannot be obtained under application of an electric field. A thickness in the range of 5 nm to 10 μm is suitable, and a thickness in the range of 10 nm to 0.2 μm is preferable.

In the wet process, the materials forming each layer are dissolved or dispersed in a suitable solvent such as ethanol, chloroform, tetrahydrofuran and dioxane, and a thin film is formed from the solution or the dispersion. Any of the above solvents can be used. Suitable resins and additives may be used in any of the organic thin films to improve the property for film formation and prevent formation of pin holes. Examples of the resin which can be used include insulating resins such as polystyrene, polycarbonates, polyarylates, polyesters, polyamides, polyurethanes, polysulfones, polymethyl methacrylate, polymethyl acrylate and cellulose, copolymers of the insulating resins, photo-conductive resins such as poly-N-vinylcarbazoles and polysilanes and electrically conductive resins such as polythiophene and polypyrrol. Examples of the additive include antioxidants, ultraviolet light absorbents and plasticizers.

The organic EL device of the present invention can be utilized as a planar light emitting body such as flat panel displays of wall televisions, a back light for copiers, printers and liquid crystal displays, a light source for instruments, a display plate and a marking light. The material of the present invention can also be used in the fields other than the organic EL device such as electronic photo-sensitive materials, photo-electric converters, solar batteries and image sensors.

### EXAMPLES

The present invention will be described more specifically with reference to examples in the following.

### Synthesis Example 1 Synthesis of Compound (D-2-3)

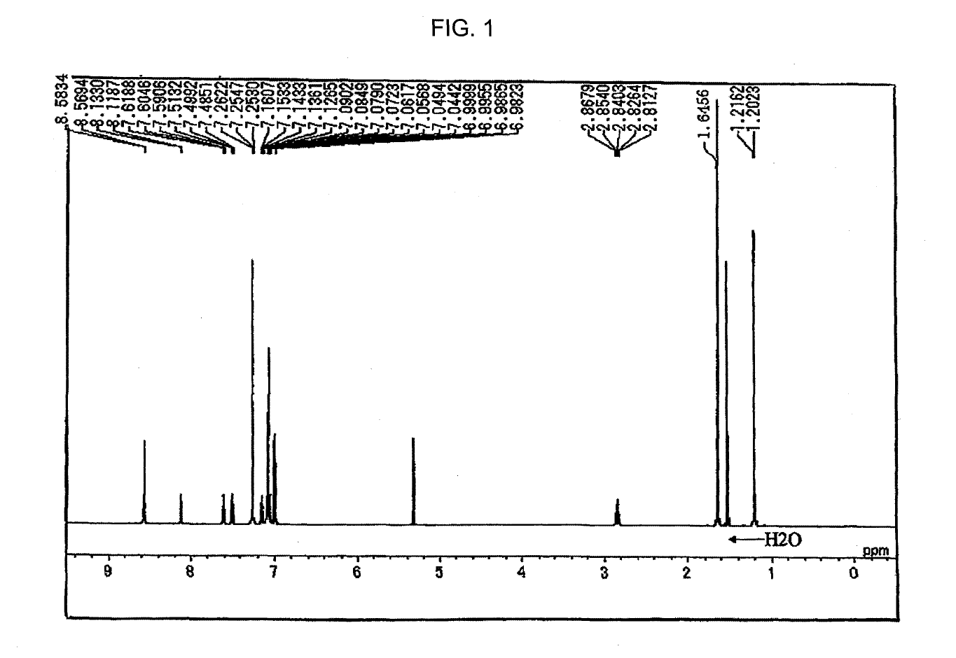

Under a stream of argon, 3.8 g (10 mmole) of 6,12-dibromochrysene, 8.2 g (25 mmole) of 4-isopropylphenyl-N-4-(2-phenylpropane)phenyl)amine, 0.03 g (1.5% by mole) of palladium acetate, 0.06 g (3% by mole) of tri-t-butylphosphine, 2.4 g (25 mmole) of t-butoxysodium and 100 ml of dry toluene were placed into a 300 ml three-necked flask equipped with a condenser, and the resultant mixture was heated at 100° C. under stirring for one night. When the reaction was completed, the formed crystals were separated by filtration and washed with 50 ml of toluene and 100 ml of methanol, and 7.0 g of a white powder substance was obtained. The obtained product was identified to be Compound (D-2-3) from the ¹H-NMR spectrum (refer to FIG. 1 and Table 1) and by the measurement in accordance with the field desorption mass spectroscopy (FD-MS) (the yield: 80%). The ¹H-NMR spectrum was obtained using DRX-500 manufactured by BRUCKER Company in a heavy methylene chloride solution. The obtained compound had a wavelength of the maximum absorption of 407 nm and a wavelength of the maximum fluorescence of 455 nm as measured in a toluene solution.

TABLE 1

| No | Position(ppm) |
|---|---|
| 1 | 1.20 |
| 2 | 1.22 |
| 3 | 1.65 |
| 4 | 2.81 |
| 5 | 2.83 |
| 6 | 2.84 |
| 7 | 2.85 |
| 8 | 2.87 |
| 9 | 6.98 |
| 10 | 6.99 |
| 11 | 7.00 |
| 12 | 7.00 |
| 13 | 7.04 |
| 14 | 7.05 |
| 15 | 7.06 |
| 16 | 7.06 |
| 17 | 7.07 |
| 18 | 7.08 |
| 19 | 7.09 |
| 20 | 7.09 |
| 21 | 7.13 |
| 22 | 7.14 |
| 23 | 7.14 |
| 24 | 7.15 |
| 25 | 7.16 |
| 26 | 7.25 |
| 27 | 7.26 |
| 28 | 7.26 |
| 29 | 7.49 |
| 30 | 7.50 |
| 31 | 7.51 |
| 32 | 7.59 |
| 33 | 7.61 |
| 34 | 7.62 |
| 35 | 8.12 |
| 36 | 8.13 |
| 37 | 8.57 |
| 38 | 8.58 |

### Synthesis Example 2 Synthesis of Compound (D-2-6)

Under a stream of argon, 3.8 g (10 mmole) of 6,12-dibromochrysene, 9.2 g (25 mmole) of 4-cyclohexylphenyl-N-4-(2-phenylpropane)phenyl)-amine, 0.03 g (1.5% by mole) of palladium acetate, 0.06 g (3% by mole) of tri-t-butylphosphine, 2.4 g (25 mmole) of t-butoxysodium and 100 ml of dry toluene were placed into a 300 ml three-necked flask equipped with a condenser, and the resultant mixture was heated at 100° C. under stirring for one night. When the reaction was completed, the formed crystals were separated by filtration and washed with 50 ml of toluene and 100 ml of methanol, and 7.6 g of a white powder substance was obtained. The obtained product was identified to be Compound (D-2-6) by the measurement in accordance with FD-MS (the yield: 80%). The obtained compound had a wavelength of the maximum absorption of 408 nm and a wavelength of the maximum fluorescence of 454 nm as measured in a toluene solution.

### Synthesis Example 3 Synthesis of Compound (D-4-1)

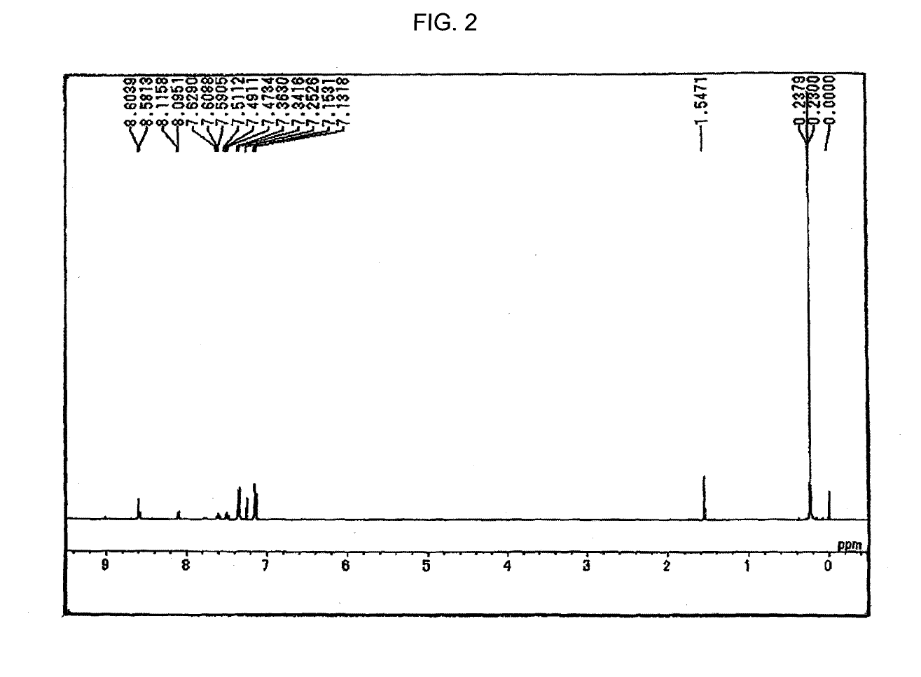

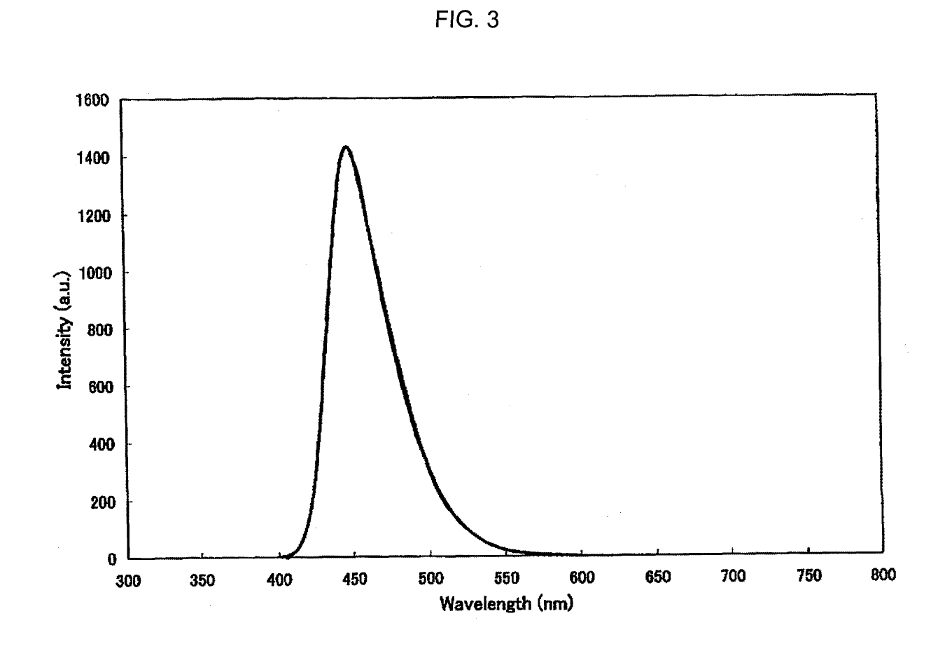

Under a stream of argon, 3.8 g (10 mmole) of 6,12-dibromochrysene, 7.8 g (25 mmole) of bis(4-trimethylsilylphenyl)amine, 0.03 g (1.5% by mole) of palladium acetate, 0.06 g (3% by mole) of tri-t-butylphosphine, 2.4 g (25 mmole) of t-butoxysodium and 100 ml of dry toluene were placed into a 300 ml three-necked flask equipped with a condenser, and the resultant mixture was heated at 100° C. under stirring for one night. When the reaction was completed, the formed crystals were separated by filtration and washed with 50 ml of toluene and 100 ml of methanol, and 5.1 g of a light yellow powder substance was obtained. The obtained product was identified to be Compound (D-4-1) from the ¹H-NMR spectrum (refer to FIG. 2) and by the measurement in accordance with FD-MS (the yield: 60%). The obtained compound had a wavelength of the maximum absorption of 402 nm and a wavelength of the maximum fluorescence of 448 nm as measured in a toluene solution (refer to FIG. 3).

### Synthesis Example 4 Synthesis of Compound (D-4-7)

Under a stream of argon, 3.8 g (10 mmole) of 6,12-dibromochrysene, 6.4 g (25 mmole) of (4-trimethylsilylphenyl)tolylamine, 0.03 g (1.5% by mole) of palladium acetate, 0.06 g (3% by mole) of tri-t-butylphosphine, 2.4 g (25 mmole) of t-butoxysodium and 100 ml of dry toluene were placed into a 300 ml three-necked flask equipped with a condenser, and the resultant mixture was heated at 100° C. under stirring for one night. When the reaction was completed, the formed crystals were separated by filtration and washed with 50 ml of toluene and 100 ml of methanol, and 5.1 g of a light yellow powder substance was obtained. The obtained product was identified to be Compound (D-4-7) by the measurement in accordance with FD-MS (the yield: 70%). The obtained compound had a wavelength of the maximum absorption of 404 nm and a wavelength of the maximum fluorescence of 450 nm as measured in a toluene solution.

### Synthesis Example 5 Synthesis of Compound (D-5-4)

Under a stream of argon, 4.5 g (10 mmole) of 2,6-di-t-butyl-9,10-dibromoanthracene, 6.4 g (25 mmole) of (4-trimethylsilylphenyl)tolyl-amine, 0.03 g (1.5% by mole) of palladium acetate, 0.06 g (3% by mole) of tri-t-butylphosphine, 2.4 g (25 mmole) of t-butoxysodium and 100 ml of dry toluene were placed into a 300 ml three-necked flask equipped with a condenser, and the resultant mixture was heated at 100° C. under stirring for one night. When the reaction was completed, the formed crystals were separated by filtration and washed with 50 ml of toluene and 100 ml of methanol, and 6.2 g of a yellow powder substance was obtained. The obtained product was identified to be Compound (D-4-7) by the measurement in accordance with FD-MS (the yield: 78%). The obtained compound had a wavelength of the maximum absorption of 455 nm and a wavelength of the maximum fluorescence of 510 nm as measured in a toluene solution.

### Example 1

On a glass substrate having a size of 25 mm×75 mm×1.1 mm thickness, a transparent electrode composed of indium tin oxide and having a thickness of 120 nm was formed. After the obtained glass substrate having the transparent electrode was cleaned by irradiation with ultraviolet light and exposure to ozone, the cleaned glass substrate was attached to a vacuum vapor deposition apparatus.

In the first step, N′,N″-bis[(4-diphenylamino)phenyl]-N′,N″-diphenylbiphenyl-4,4′-diamine was vapor deposited to form a hole injecting layer having a thickness of 60 nm, and N,N,N′,N′-tetrakis-(4-biphenyl)-4,4′-benzidine was vapor deposited on the formed layer to form a hole transporting layer having a thickness of 20 mm. Then, 10,10′-bis[1,1′,4′,1″]terphenyl-2-yl-9,9′-bianthracenyl and Compound (D-2-3) prepared above were simultaneously vapor deposited in amounts such that the ratio of the amounts by weight was 40:2, and a light emitting layer having a thickness of 40 nm was formed.

As the electron injecting layer, tris(8-hydroxyquinolinato)aluminum was vapor deposited to form a layer having a thickness of 20 nm. Lithium fluoride was vapor deposited to form a layer having a thickness of 1 nm, and then aluminum was vapor deposited to form a layer having a thickness of 150 nm. The formed aluminum/lithium fluoride layer worked as the cathode. An organic EL device was prepared as described above.

The prepared device was examined by passing an electric current. Blue light (the maximum wavelength of the emitted light: 461 nm) was emitted at an efficiency of light emission of 6.7 cd/A and a luminance of emitted light of 670 cd/cm² under a voltage of 6.5 V and a current density of 10 mA/cm². When the device was examined by continuously passing a direct electric current at an initial luminance of 500 cd/cm², the half life was 10,000 hours or longer.

### Example 2

An organic EL device was prepared in accordance with the same procedures as those conducted in Example 1 except that Compound (D-2-6) was used in place of Compound (D-2-3).

The prepared device was examined by passing an electric current. Blue light (the maximum wavelength of the emitted light: 460 nm) was emitted at an efficiency of light emission of 6.5 cd/A and a luminance of emitted light of 650 cd/cm² under a voltage of 6.5 V and a current density of 10 mA/cm². When the device was examined by continuously passing a direct electric current at an initial luminance of 500 cd/cm², the half life was 10,000 hours or longer.

### Example 3

An organic EL device was prepared in accordance with the same procedures as those conducted in Example 1 except that Compound (D-1-8) was used in place of Compound (D-2-3).

The prepared device was examined by passing an electric current. Green light (the maximum wavelength of the emitted light: 525 nm) was emitted at an efficiency of light emission of 19.5 cd/A and a luminance of emitted light of 1,950 cd/cm² under a voltage of 6.5 V and a current density of 10 mA/cm². When the device was examined by continuously passing a direct electric current at an initial luminance of 500 cd/cm², the half life was 100,000 hours or longer.

### Comparative Example 1

An organic EL device was prepared in accordance with the same procedures as those conducted in Example 1 except that 6,12-bis(4-isopropylphenyl-p-tolylamino)chrysene was used in place of Compound (D-2-3).

The prepared device was examined by passing an electric current. Blue light (the maximum wavelength of the emitted light: 462 nm) was emitted at an efficiency of light emission of 5.9 cd/A and a luminance of emitted light of 594 cd/cm² under a voltage of 6.3 V and a current density of 10 mA/cm². When the device was examined by continuously passing a direct electric current at an initial luminance of 500 cd/cm², the half life was 4,590 hours.

It is shown by the above results that the material for organic EL devices which is substituted with a substituent having the benzene ring at the end portion could prevent association of molecules of the compounds unlike compounds having no substituents described above, and the half life could be increased.

### Comparative Example 2

In the same procedures as those conducted in Example 1, 2,6-cyclohexyl-N,N—N′,N′-tetrakis(4-(2-phenylpropan-2-yl)-phenyl)anthracene-9,10-diamine was used in place of Compound (D-2-3). When the above compound was heated in the vacuum vapor deposition apparatus, formation of decomposition products was observed. Therefore, the obtained material could not be used as the material for organic EL devices.

### Example 4

An organic EL device was prepared in accordance with the same procedures as those conducted in Example 1 except that 10-(4-(naphthalen-1-yl)phenyl)-9-(naphthalen-2-yl)anthracene was used in place of 10,10′-bis[1,1′,4′,1″-]terphenyl-2-yl-9,9′-bianthracenyl and Compound (D-4-1) was used in place of Compound (D-2-3).

The prepared device was examined by passing an electric current. Pure blue light (the maximum wavelength of the emitted light: 452 nm) was emitted at an efficiency of light emission of 3.0 cd/A and a luminance of emitted light of 300 cd/cm² under a voltage of 6.5 V and a current density of 10 mA/cm².

### Example 5

An organic EL device was prepared in accordance with the same procedures as those conducted in Example 4 except that Compound (D-4-6) was used in place of Compound (D-4-1).

The prepared device was examined by passing an electric current. Pure green light (the maximum wavelength of the emitted light: 505 nm) was emitted at an efficiency of light emission of 3.0 cd/A and a luminance of emitted light of 300 cd/cm² under a voltage of 6.5 V and a current density of 10 mA/cm².

### INDUSTRIAL APPLICABILITY

As described in detail in the above, the organic EL device using the material for organic EL devices of the present invention provides a sufficient luminance of emitted light under application of a low voltage and a great efficiency of light emission, suppresses degradation and has a long life. Therefore, the organic EL device can be used as the light source for planar light emitting products such as wall televisions and the back light for displays.

1. A material for organic electroluminescence devices which comprises an aromatic amine derivative represented by following general formula (1) or general formula (2): 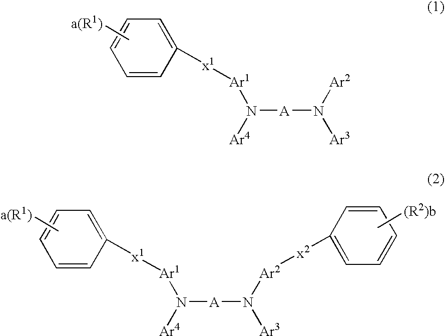
wherein
A represents a substituted or unsubstituted aromatic hydrocarbon cyclic group having 6 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;
Ar¹ to Ar⁴ each independently represent a substituted or unsubstituted aromatic hydrocarbon cyclic group having 6 to 40 nuclear carbon atoms or a substituted or unsubstituted aromatic heterocyclic group having 5 to 40 nuclear carbon atoms, wherein Ar¹ represents a divalent group, Ar² represents a monovalent or a divalent group and Ar³ and Ar⁴ each represent a monovalent group;
X¹ to X⁴ each independently represent —O—, —S—, >C═O, >SO₂, —(CₓH₂ₓ)—O—(C_{y}H_{2y})—, x and y each representing an integer of 0 to 20 excluding a case where x+y=0, a substituted or unsubstituted alkylidene group having 2 to 20 carbon atoms, a substituted or unsubstituted alkylene group having 2 to 20 carbon atoms or a substituted or unsubstituted divalent aliphatic cyclic group having 3 to 10 nuclear carbon atoms;
R¹ and R² each independently represent hydrogen atom, a substituted or unsubstituted alkyl group having 1 to 50 carbon atoms, a substituted or unsubstituted aryl group having 5 to 50 nuclear carbon atoms, a substituted or unsubstituted aralkyl group having 6 to 50 nuclear carbon atoms, a substituted or unsubstituted cycloalkyl group having 3 to 50 nuclear carbon atoms, a substituted or unsubstituted alkoxyl group having 1 to 50 carbon atoms, a substituted or unsubstituted aryloxyl group having 5 to 50 nuclear carbon atoms, a substituted or unsubstituted arylamino group having 5 to 50 nuclear carbon atoms, a substituted or unsubstituted alkylamino group having 1 to 20 carbon atoms or a substituted or unsubstituted heterocyclic group having 5 to 50 nuclear carbon atoms; and
a and b each represent an integer of 1 to 5 and, when any of a and b represents an integer of 2 or greater, groups in ( ) may be same with or different from each other, and adjacent groups among the groups represented by R¹ or R² may be bonded to each other to form a cyclic structure.

2. A material for organic electroluminescence devices according to claim 1, which comprises an aromatic amine derivative represented by following general formula (3) or general formula (4): 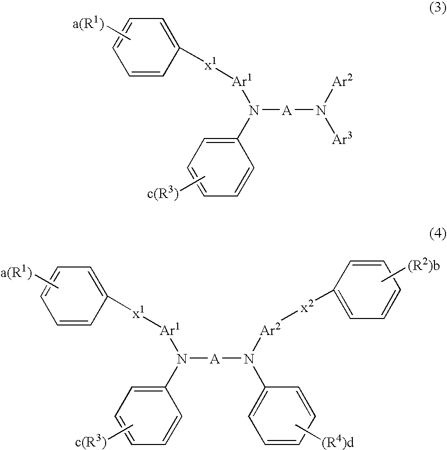
wherein
A and Ar¹ to Ar³ are as defined in claim 1, and R¹ to R⁴ are as defined for R¹ and R² in claim 1; and
a to d each represent an integer of 1 to 5 and, when any of a to d represents an integer of 2 or greater, groups in ( ) may be same with or different from each other, and adjacent groups among the groups represented by R¹ to R⁴ may be bonded to each other to form a cyclic structure.

3. A material for organic electroluminescence devices according to claim 2, which comprises an aromatic amine derivative represented by general formula (3) or general formula (4) in which R³ represents a secondary or tertiary alkyl group.

4. A material for organic electroluminescence devices according to claim 2, which comprises an aromatic amine derivative represented by general formula (3) or general formula (4) in which c represents an integer of 2 to 3.

5. A material for organic electroluminescence devices which comprises an aromatic amine derivative represented by following general formula (5): 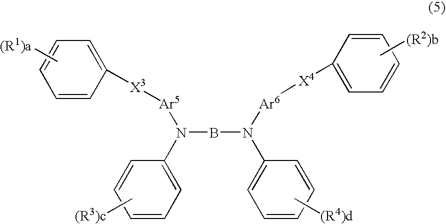
wherein
B represents a substituted or unsubstituted aromatic hydrocarbon cyclic group having 10 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;
Ar⁵ and Ar⁶ each independently represent a substituted or unsubstituted divalent aromatic hydrocarbon cyclic group having 6 to 40 nuclear carbon atoms;
X³ and X⁴ each independently represent a group represented by following formula: 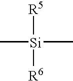
R⁵ and R⁶ each independently representing hydrogen atom, a substituted or unsubstituted alkyl group having 1 to 10 carbon atoms or a substituted or unsubstituted aryl group having 5 to 20 nuclear carbon atoms;
R¹ to R⁴ each independently represent hydrogen atom, a substituted or unsubstituted alkyl group having 1 to 50 carbon atoms, a substituted or unsubstituted aryl group having 5 to 50 nuclear carbon atoms, a substituted or unsubstituted aralkyl group having 6 to 50 nuclear carbon atoms, a substituted or unsubstituted cycloalkyl group having 3 to 50 nuclear carbon atoms, a substituted or unsubstituted alkoxyl group having 1 to 50 carbon atoms, a substituted or unsubstituted aryloxyl group having 5 to 50 nuclear carbon atoms, a substituted or unsubstituted arylamino group having 5 to 50 nuclear carbon atoms, a substituted or unsubstituted alkylamino group having 1 to 20 carbon atoms or a substituted or unsubstituted heterocyclic group having 5 to 50 nuclear carbon atoms; and
a to d each represent an integer of 1 to 5 and, when any of a to d represents an integer of 2 or greater, groups in ( ) may be same with or different from each other, and adjacent groups among the groups represented by R¹ to R⁴ may be bonded to each other to form a cyclic structure.

6. A material for organic electroluminescence devices which comprises an aromatic amine derivative represented by following general formula (6): 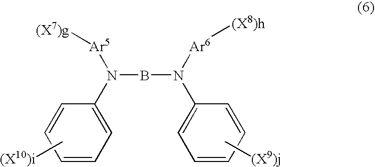
wherein
B represents a substituted or unsubstituted aromatic hydrocarbon cyclic group having 10 to 40 nuclear carbon atoms, a substituted or unsubstituted aromatic heterocyclic group having 5 to 40 nuclear carbon atoms or a divalent group comprising 2 to 10 cyclic structural units which are groups of a same type or different types selected from the aromatic hydrocarbon cyclic group and the aromatic heterocyclic group and are bonded to each other directly or via at least one of oxygen atom, nitrogen atom, sulfur atom, chain structural units and aliphatic cyclic groups, wherein the chain structural unit and the aliphatic cyclic group have 1 to 20 nuclear carbon atoms and may have heteroatoms;
Ar⁵ and Ar⁶ each independently represent a substituted or unsubstituted divalent aromatic hydrocarbon cyclic group having 6 to 40 nuclear carbon atoms;
X⁷ to X¹⁰ each independently represent a group represented by following formula: 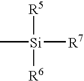
R⁵ to R⁷ each independently representing hydrogen atom, a substituted or unsubstituted alkyl group having 1 to 10 carbon atoms or a substituted or unsubstituted aryl group having 5 to 20 nuclear carbon atoms; and
g, h, i and j each represent an integer of 0 or 1, and a case where all of g to j represent 0 is excluded.

7. A material for organic electroluminescence devices according to claim 6, wherein all of g, h, i and j represent 1 in general formula (6).

8. A material for organic electroluminescence devices according to claim 6, wherein B represents a divalent residue group of a substituted or unsubstituted naphthalene, anthracene, pyrene or chrysene in general formula (6).

9. A material for organic electroluminescence devices according to any one of claims 1, 5 and 6, which is a doping material for an organic electroluminescence device.

10. An organic electroluminescence device comprising an anode, a cathode and an organic thin film layer which comprises a single layer or a plurality of layers comprising at least a light emitting layer and is disposed between the anode and the cathode, wherein at least one layer in the organic thin film layer comprises the material for organic electroluminescence devices described in any one of claims 1, 5 and 6 singly or as a component of a mixture.

11. An organic electroluminescence device according to claim 10, wherein the light emitting layer comprises the material for organic electroluminescence devices singly or as a component of a mixture.

12. An organic electroluminescence device according to claim 10, wherein the light emitting layer comprises 0.1 to 20% by weight of the material for an organic electroluminescence.

13. An organic electroluminescence device according to claim 10, wherein the light emitting layer comprises the material for organic electroluminescence devices as a doping material and, as a host material, an anthracene derivative represented by following general formula (7): 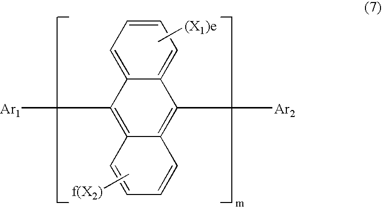
wherein X₁ and X₂ each independently represent hydrogen atom, a substituted or unsubstituted alkyl group having 1 to 50 carbon atoms, a substituted or unsubstituted aryl group having 5 to 50 carbon atoms, a substituted or unsubstituted aralkyl group having 1 to 50 carbon atoms, a substituted or unsubstituted cycloalkyl group having 5 to 50 carbon atoms, a substituted or unsubstituted alkoxyl group having 1 to 50 carbon atoms, a substituted or unsubstituted aryloxyl group having 5 to 50 carbon atoms, a substituted or unsubstituted arylamino group having 5 to 50 carbon atoms, a substituted or unsubstituted alkylamino group having 1 to 20 carbon atoms, a substituted or unsubstituted heterocyclic group having 5 to 50 carbon atoms or a halogen atom, e and f each independently represent an integer of 0 to 4 and atoms and, when any of e and f represent an integer of 2 or greater, a plurality of atoms and groups represented by X₁ and X₂ may be same with or different from each other;
Ar₁ and Ar₂ each independently represent a substituted or unsubstituted aryl group having 5 to 50 carbon atoms or a substituted or unsubstituted heterocyclic group having 5 to 50 carbon atoms, and at least one of Ar₁ and Ar₂ represents a substituted or unsubstituted condensed ring aryl group having 10 to 50 nuclear carbon atoms or a substituted or unsubstituted aryl group having 10 or more carbon atoms; and
m represents an integer of 1 to 3, and groups in [ ] may be same with or different from each other when m represents an integer of 2 or greater.

14. An organic electroluminescence device according to claim 10, wherein the light emitting layer comprises the material for organic electroluminescence devices as a doping material and, as a host material, an anthracene derivative represented by following general formula (8): 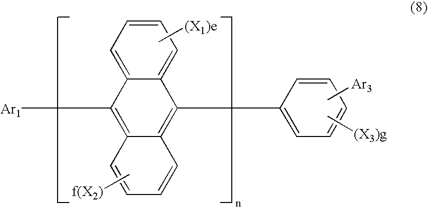
wherein X₁ and X₂ each independently represent hydrogen atom, a substituted or unsubstituted alkyl group having 1 to 50 carbon atoms, a substituted or unsubstituted aryl group having 5 to 50 carbon atoms, a substituted or unsubstituted aralkyl group having 1 to 50 carbon atoms, a substituted or unsubstituted cycloalkyl group having 5 to 50 carbon atoms, a substituted or unsubstituted alkoxyl group having 1 to 50 carbon atoms, a substituted or unsubstituted aryloxyl group having 5 to 50 carbon atoms, a substituted or unsubstituted arylamino group having 5 to 50 carbon atoms, a substituted or unsubstituted alkylamino group having 1 to 20 carbon atoms, a substituted or unsubstituted heterocyclic group having 5 to 50 carbon atoms or a halogen atom, e and f each independently represent an integer of 0 to 4, and atoms and, when any of e and f represent an integer of 2 or greater, a plurality of atoms and groups represented by X₁ and X₂ may be same with or different from each other;
Ar₁ represents a substituted or unsubstituted condensed ring aryl group having 10 to 50 nuclear carbon atoms, and Ar₃ represents a substituted or unsubstituted aryl group having 5 to 50 nuclear carbon atoms; and
n represents an integer of 1 to 3, and groups in [ ] may be same with or different from each other when n represents an integer of 2 or greater.

15. An organic electroluminescence device according to claim 10, wherein the light emitting layer comprises the material for organic electroluminescence devices as a doping material and, as a host material, a pyrene derivative represented by following general formula (9): 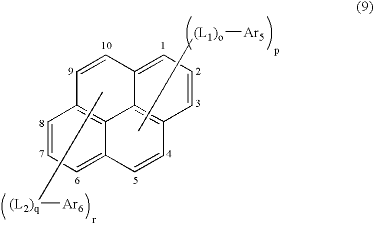
wherein
Ar₅ and Ar₆ each independently represent a substituted or unsubstituted aryl group having 5 to 50 nuclear carbon atoms, and L₁ and L₂ each represent a substituted or unsubstituted phenylene group, a substituted or unsubstituted naphthalenylene group, a substituted or unsubstituted fluorenylene group or a substituted or unsubstituted dibenzosilolylene group;
o represents an integer of 0 to 2, p represents an integer of 1 to 4, q represents an integer of 0 to 2, and r represents an integer of 1 to 4;
the group represented by L₁ or Ar₅ is bonded at one of 1 to 5 positions of pyrene, and the group represented by L₂ or Ar₆ is bonded at one of 6 to 10 positions of pyrene; and
when p+r is an even number, the groups represented by Ar₅, Ar₆, L₁ and L₂ satisfy the following condition (1) or (2):
(1) Ar₅ and Ar₆ represent groups different from each other and/or L₁ and L₂ represent groups different from each other;
(2) when Ar₅ and Ar₆ represent a same group and L₁ and L₂ represent a same group,
(2-1) o and q represent integers different from each other and/or p and r represent integers different from each other, or
(2-2) when o and q represent a same integer and p and r represent a same integer,
a case where positions of substitution of the groups represented by L₁ and L₂ or Ar₅ and Ar₆ on pyrene are 1-position and 6-position, respectively, or 2-position and 7-position, respectively, is excluded when
(2-2-1) the groups represented by L₁ and L₂ or two positions on pyrene are bonded at different bonding positions on the groups represented by Ar₅ and Ar₆, respectively, or
(2-2-2) the groups represented by L₁ and L₂ or two positions on pyrene are bonded at same bonding position on the groups represented by Ar₅ and Ar₆, respectively.
